(19) United States
(12) Patent Application Publication
(10) Pub. No.: US 2019/0276481 A1
(43) Pub. Date: Sep. 12, 2019

(54) Liver Prodrugs of Mitochondrial Proton Ionophores

(71) Applicant: NEUROVIVE PHARMACEUTICAL AB, Lund (SE)

(72) Inventors: Magnus Joakim Hansson, Landskrona (SE); Eskil Elmer, Lund (SE); Matthew Alan Gregory, Bourn (GB); Steven James Moss, Balsham (GB)

(21) Appl. No.: 16/348,585

(22) PCT Filed: Nov. 17, 2017
(86) PCT No.: PCT/EP2017/079548

§ 371 Date: May 9, 2019

(30) Foreign Application Priority Data
Nov. 18, 2016 (DK) PA 2016 70919

(51) Int. Cl. C07F 9/24; A61P 1/16

(52) U.S. Cl. CPC C07F 9/2458; C07F 9/242; C07F 9/247; A61P 1/16

(57) ABSTRACT

The present invention provides novel liver-targeted prodrugs of mitochondrial proton ionophores. These compounds have utility in medicine including their use in treatment of diseases such as NASH and NAFLD.

### FIELD OF THE INVENTION

The present invention provides novel liver-metabolised prodrugs of mitochondrial proton ionophores (protonophores). These compounds are cleaved from an inactive non-uncoupling form in the liver to release mild uncoupling agents capable of causing mild mitochondrial uncoupling, with potential in treatment of Non-alcoholic steatohepatitis (NASH) and/Non-alcoholic fatty liver disease (NAFLD). The invention also relates to their use in medicine notably in the treatment of Non-alcoholic fatty liver disease (NAFLD) and Non-alcoholic steatohepatitis (NASH). The invention also relates to the specific use of salicylanilide in medicine notably in the treatment of Non-alcoholic fatty liver disease (NAFLD) and Non-alcoholic steatohepatitis (NASH).

### BACKGROUND OF THE INVENTION

Non-alcoholic fatty liver disease (NAFLD) affect up to 30% of the world's population and is an important step towards development of Non-alcoholic steatohepatitis (NASH).

However, attempts to reduce the incidence of NAFLD with pharmacologic agents has been met with limited success.

Non-alcoholic fatty liver disease (NAFLD) is the most common cause of referral to liver clinics, and its progressive form, non-alcoholic steatohepatitis (NASH), can lead to cirrhosis and end-stage liver disease. Mitochondrial protonophores, such as dinitrophenol (DNP) have long been known to promote weight loss and impact markers of NAFLD and NASH in preclinical models. However, despite their potential, their development has been limited due to their toxicity. The aim of this study was to explore a new class of liver targeted protonophores for in vitro uncoupling activity and suitability as potential treatment of NAFLD and NASH.

Mitochondrial proton ionophores or uncouplers, such as 2,4 dinitrophenol (DNP), have long been known to promote weight loss. However, safety concerns led to it being one of the first agents banned by the FDA. Acute administration of 20-50 mg/kg body weight can be lethal (Hsaio et al., 2005 Clin Toxicol (Phila). 43 (4): 281-285), with the major acute toxicity coming from hyperthermia, through uncoupling in muscle tissue (Simkins, 1937 J Am Med Assoc. 108: 2110-2117). Chronic toxicities can include cataracts, bone marrow, CNS and CVS side effects (Public Health Service, U.S. Department of Health and Human Services (1995). “Toxicological Profile for Dinitrophenols”. Agency for Toxic Substances and Disease Registry) (Bushke 1947, American Journal of Ophthalmology Volume 30, Issue 11, November 1947, Pages 1356-1368).

Both DNP in drinking water (Goldgof et al., 2014 J Biol Chem. 2014 Jul. 11; 289(28): 19341-19350) and controlled release formulations of DNP have been shown to have potential in treatment of NAFLD and related diseases. Daily administrations reversed NAFLD, insulin resistance, T2D, NASH, and liver fibrosis in rats without detectable toxicity (Perry et al., 2015 Science. 2015 Mar. 13; 347(6227): 1253-1256). However, this treatment required very careful monitoring and dose adjustment to maintain plasma concentrations of DNP in the range 1-5 uM and avoid toxicity.

Other uncouplers have also shown promise, such as salsalate, which was seen to stimulate brown adipose tissue respiration independent of UCP1 (Smith et al., 2016, Diabetes 2016 November; 65(11): 3352-3361).

Simple ether prodrugs of DNP have also been described (WO2015/031598).

Salicylanilide, also known as 2-Hydroxy-N-phenylbenzamide, is used as a topical antifungal and fungicide (U.S. Pat. No. 2,485,339). Substituted salicylanilides, have been shown to have uncoupling activity (See S13 in Terada 1990, Environ Health Perspect. 1990 July; 87: 213-218). However, the vast majority of therapeutic development (especially as antihelminthics) has been on substituted salicylanilides (such as S13, niclosamine, oxyclozanide and rafoxanide) which have been developed as antihelminthic drugs (Swan JI S.Afr.vet.Ass. (1999) 70(2): 61-70).

We have discovered that efficient liver targeted release of protonophores can be generated via a phosphate prodrug chemistry where the cleavage mechanism is triggered by metabolic enzymes significantly more prevalent in the liver. It is advantageous to target the protonophore moiety and uncoupling activity to liver, which leads to a positive effect on liver metabolism, NAFLD or NASH, versus activity in other organs, which could lead to toxicity (such as hyperthermia).

We have also discovered that salicylanilide is a potent, low toxicity protonophore with suitable properties and has significant potential for treatment of NASH and/or NAFLD, diabetes and/or weight loss. In particular, it has high permeability, oral bioavailability and is natural liver-targeted after oral dosing. These properties are all advantageous for an agent to treat NAFLD or NASH, especially with respect to focussing exposure to the target organs and reducing toxicity to other organs. Thus, in some of the compounds of the invention, the salicylanilide structure is part of the structure.

In addition, non-nitro containing protonophore moieties may be advantageous as they may lead to a reduction in toxicity, such as a reduction in the development of cataracts.

Accordingly, there is a need of providing liver targeted prodrugs of proton ionophores with improved properties to treat NAFLD and/or NASH.

### DESCRIPTION OF THE INVENTION

The present invention describes liver targeted prodrugs of protonophores. These have no or limited uncoupling activity in their dosed state, but are cleaved by liver enzymes, such as those found in microsomes to generate active uncouplers.

One advantage of the compounds of the invention is therefore their reduced uncoupling activity in the dosed state versus the form released following liver metabolism. Another advantage of the compounds of the invention is their improved tolerability. Other advantages include increased liver metabolism and reduced plasma or muscle metabolism.

The present invention provides a prodrug of Formula (I)

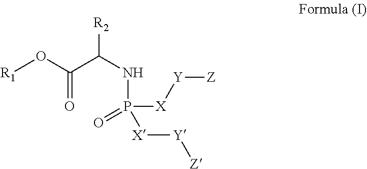

wherein:
X and X′ can independently be NH or O
Y is absent, —CR₃R₄O—, —C(═O)O—, or

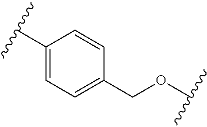 [SMILES: *OCc1ccc(*)cc1]

(X is phenyl substituent, Z connects to O)
Y′ is absent, —CR₃R₄O—, —C(═O)O—, or

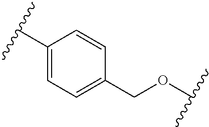 [SMILES: *OCc1ccc(*)cc1]

(X′ is phenyl substituent, Z′ connects to O)
Z is formula (II)
Z′ is CHR₂′(C═O)OR₁′, Me, Et, iPr, Ph or formula (II)
R₁ and R₁′ are independently Me, Et, iPr, nPr, tBu, iBu, sBu or CH₂CMe₃
R₂ and R₂′ are independently H, Me, Et, iPr, Ph, Bn

### R₃ is H, Me, Et

### R₄ is H, Me, Et

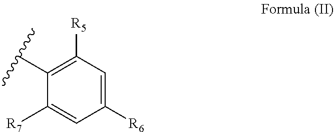

wherein:
R₅ is H, NO₂ or

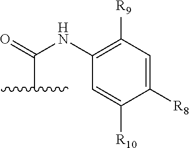

R₆ is H, NO₂, Cl, Br or I
R₇ is H, Me, Et, iPr, tBu, sBu, iBu, Cl, Br or I
R₈ is H, NO₂, Cl, Br, C(CN)H(C₆H₄)-p-Cl
R₉ is H, Cl, OH or CH₃

### R₁₀ is H or Cl

R₅ and R₆ cannot both be H;
when R₆ is Cl, R₅ cannot be H or NO₂;
when Z′ is CHR₂′(C═O)OR₁′, Me, Et, iPr then Y′ must be absent;
when Z′ is CHR₂′(C═O)OR₁′ then X′ must be NH;
when Z′ is Me, Et or iPr then X′ must be O;
when Z is Formula II and R₆ is NO₂ then Y cannot be absent
when Z′ is Formula II and R₆ is NO₂ then Y′ cannot be absent
when Z is formula II and R₆ is NO₂ and Z′ is CHR₂′(C═O)OR₁′ then R₂ and R₂, cannot be

### H or Me;

or a pharmaceutically acceptable salt thereof.

In an embodiment Z and/or Z′ are formula (II) and R₅ is

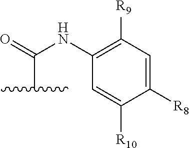

In a preferred embodiment Z and/or Z′ are formula (II) and R₅ is

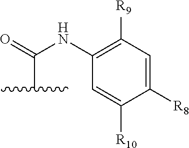

and R₆, R₇, R₈, R₉ and R₁₀ are all H.

In a preferred embodiment Z and/or Z′ are formula (II) and R₅ is

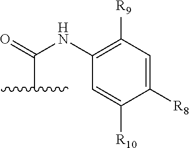

and R₆ is Cl, R₇ is H or tBu, R₈ is Cl, R₉ is NO₂ and R₁₀ is H.

In an embodiment Z′ is CHR₂′(C═O)OR₁′ and Z is formula (II) and R₅ is

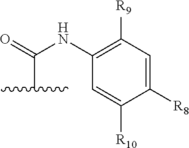

In an embodiment Z′ is CHR₂′(C═O)OR₁′, R₁ and R₁′ are iPr and R₂ and R₂′ are Me or Bn and Z is formula (II) and R₅ is

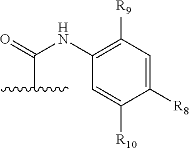

and R₆, R₇, R₈, R₉ and R₁₀ are all H.

In an embodiment Z′ is CHR₂′(C═O)OR₁′, R₁ and R₁′ are CH₂tBu and R₂ and R₂′ are Me or Bn and Z is formula (II) and R₅ is

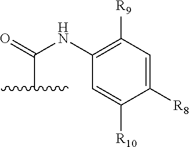

and R₆, R₇, R₈, R₉ and R₁₀ are all H.

In an embodiment Z′ is CHR₂′(C═O)OR₁′, R₁ and R₁′ are iPr and R₂ and R₂′ are Me or Bn and Z is formula (II) and R₅ is

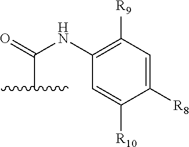

and R₆ is Cl, R₇ is H or tBu, R₈ is Cl, R₉ is NO₂ and R₁₀ is H.

In an embodiment Z′ is CHR₂′(C═O)OR₁′, R₁ and R₁′ are CH₂tBu and R₂ and R₂′ are Me or Bn and Z is formula (II) and R₅ is

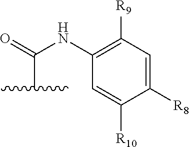

and R₆ is Cl, R₇ is H or tBu, R₈ is Cl, R₉ is NO₂ and R₁₀ is H.

Suitable embodiments include

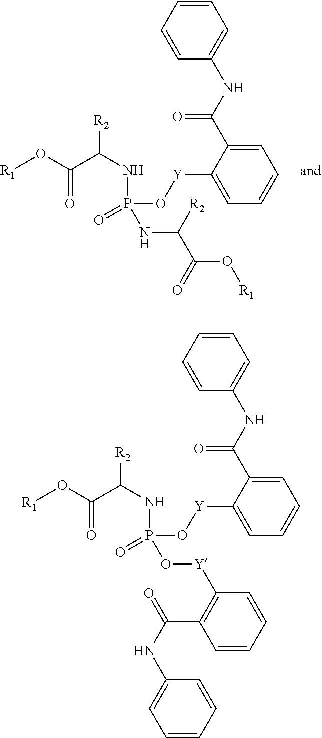

The compound may be selected from the following:

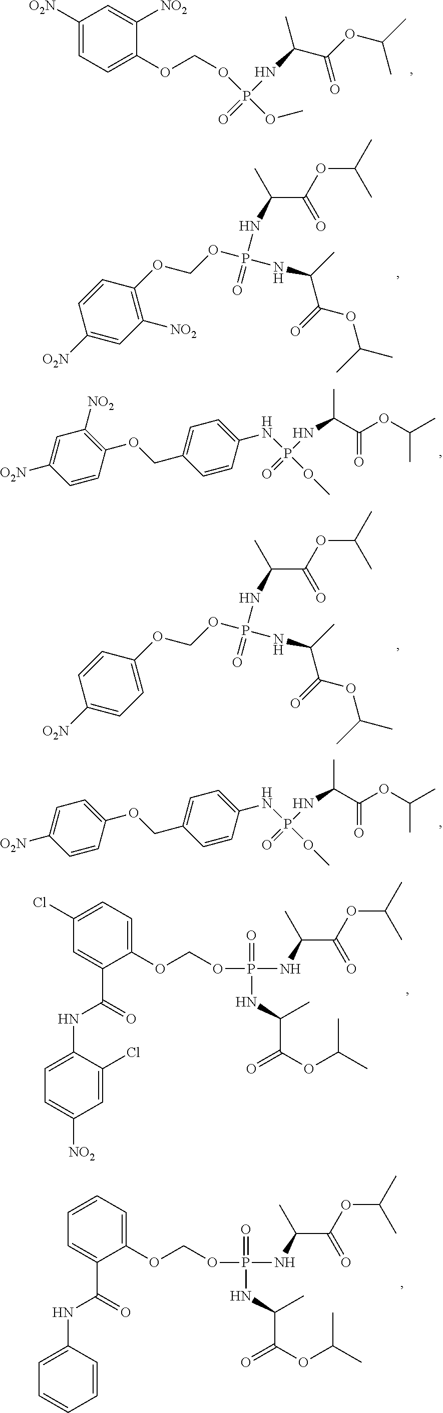

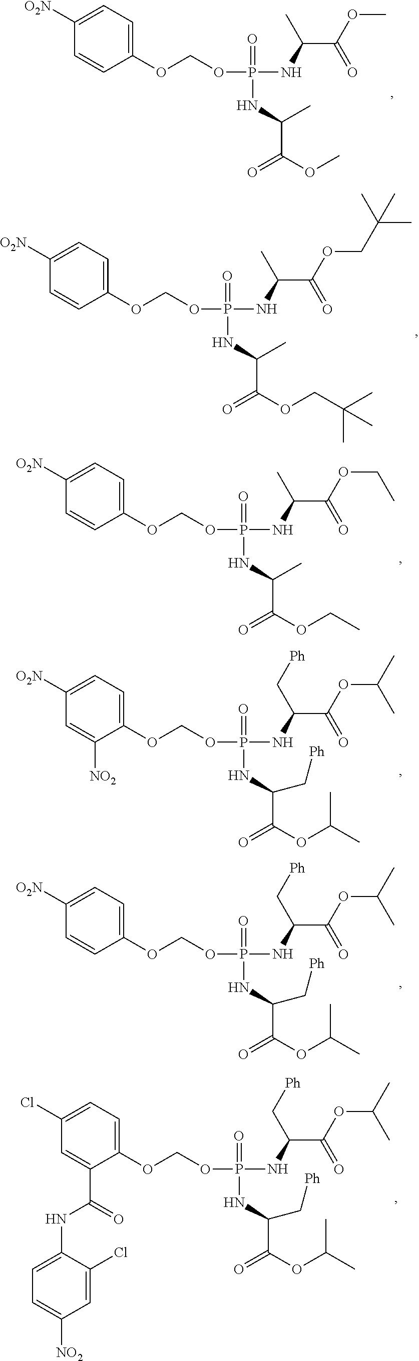

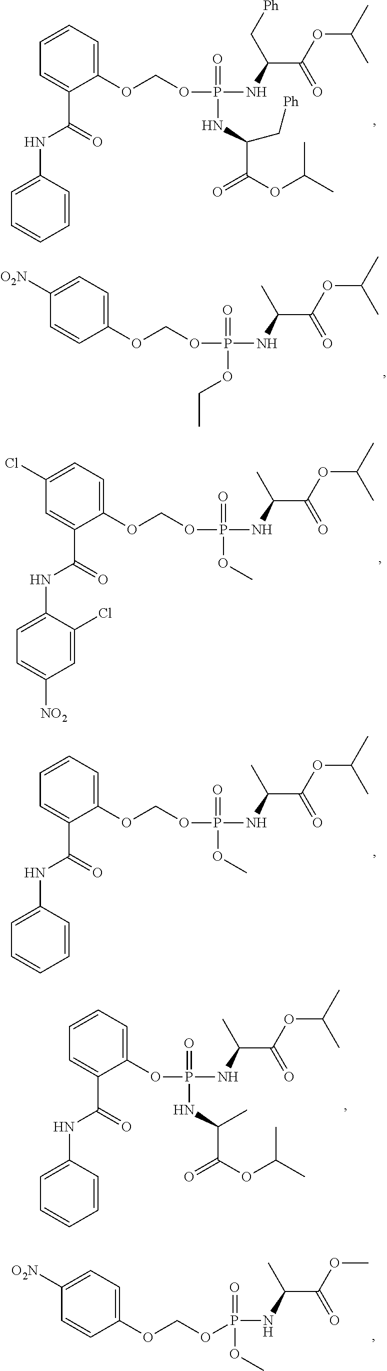

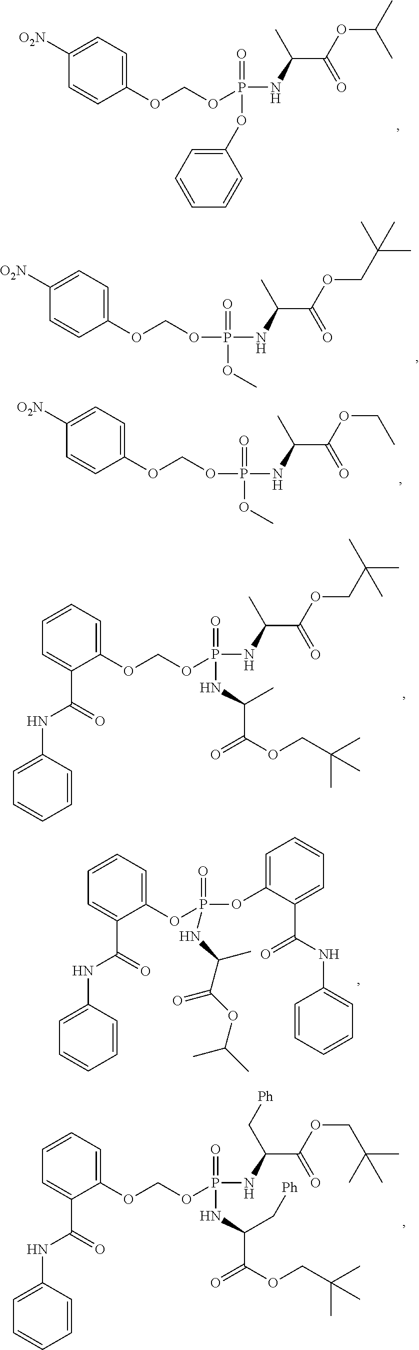

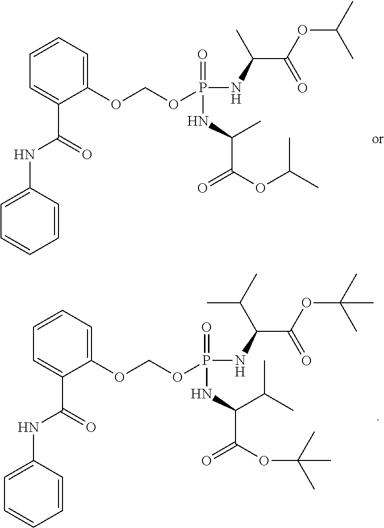 [SMILES: CC(C)OC(=O)[C@H](C)NP(=O)(N[C@@H](C)C(=O)OC(C)C)OCOc1ccccc1C(=O)Nc1ccccc1 ; CC(C)[C@H](NP(=O)(N[C@H](C(=O)OC(C)(C)C)C(C)C)OCOc1ccccc1C(=O)Nc1ccccc1)C(=O)OC(C)(C)C]

or from

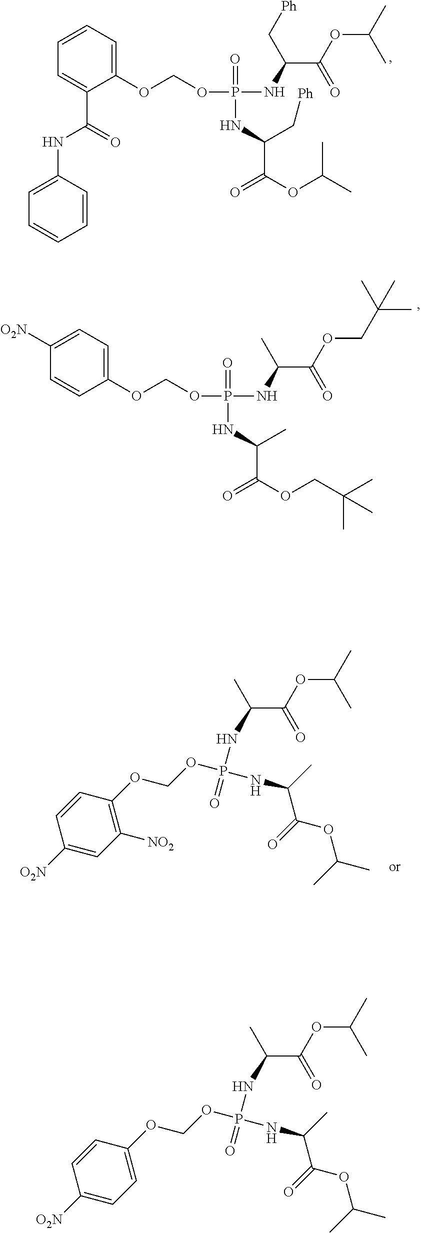 [SMILES: CC(C)OC(=O)[C@H](Cc1ccccc1)NP(=O)(N[C@@H](Cc1ccccc1)C(=O)OC(C)C)OCOc1ccccc1C(=O)Nc1ccccc1 ; C[C@H](NP(=O)(N[C@@H](C)C(=O)OCC(C)(C)C)OCOc1ccc([N+](=O)[O-])cc1)C(=O)OCC(C)(C)C ; CC(C)OC(=O)[C@H](C)NP(=O)(N[C@@H](C)C(=O)OC(C)C)OCOc1ccc([N+](=O)[O-])cc1[N+](=O)[O-] ; CC(C)OC(=O)[C@H](C)NP(=O)(N[C@@H](C)C(=O)OC(C)C)OCOc1ccc([N+](=O)[O-])cc1]

Compounds according to the present invention can be used in medicine to treat disease or disorders or they can be used in r medical research. The compounds can be used in the prevention or treatment of disorders or diseases where liver targeted mitochondrial uncoupling is useful, such as NAFLD or NASH.

The present invention also provides methods for use of salicylanilide in the prevention or treatment of disorders or diseases where liver targeted mitochondrial uncoupling is useful, such as NAFLD or NASH.

If some of the compounds disclosed herein are already known they are hereby disclaimed; thus the invention relates to the compounds as such provided that they are novel. The invention relates to the compounds disclosed herein for use in medicine, notably in the treatment of NASH or NAFLD. Other uses of the compounds appear from the description herein.

Indications for which the disclosed compounds of the invention may be therapeutically effective include Non-alcoholic fatty liver disease (NAFLD) and non-alcoholic steatohepatitis (NASH).

Methods of treating a disease in a patient provided by the present disclosure comprise administering to a patient in need of such treatment a suitable dose of one or more compounds of the invention.

An appropriate dose of a compound of the invention may be determined based on several factors, including, for example, the potency of the compound to be used, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

In addition, compared with DNP or other prodrugs of DNP, such as those described in WO2015/031598, the compounds are contemplated to show improved properties for treatment of these and related diseases, including improved tolerability, increased therapeutic index, increased ratio of liver uncoupling versus extra hepatic uncoupling and increased rate of liver prodrug metabolism versus extra hepatic prodrug metabolism.

Thus, the advantageous properties of the compound of the invention may include one or more of the following:
Increased relative liver exposure of protonophore moiety
Reduced muscle exposure of protonophore moiety
Reduced protonophore activity of parent compound
Reduced inter-patient variability
Reduced side effects
Increased therapeutic index
Reduced maximal uncoupling effect
Reduced kidney and brain exposure

### General Chemistry Methods

The skilled person will recognise that the compounds of the invention may be prepared, in known manner, in a variety of ways. The routes below are merely illustrative of some methods that can be employed for the synthesis of compounds of formula (I) that will be apparent to one skilled in the art.

For compounds where X′ is O, Y′ is bond and Z′ is alkyl such as

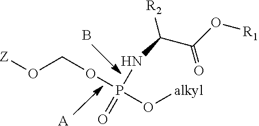

Then the principle connections required are A and B as shown.

Connection A is made by reacting two substances such as

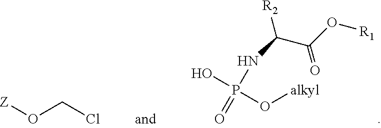

This can be done in the presence of base, such as K₂CO₃ in non-nucleophilic solvent, such as acetonitrile and in the presence of iodide to activate the C—Cl bond.

Compounds such as ZOCH₂Cl can be made by, for example, reacting a phenol (such as DNP or salicylanilide) with chloromethanesulfonyl chloride. Suitably the reaction could be performed in a biphasic system (e.g. DCM and water) with base (NaHCO₃) and a phase transfer agent (nBu₄NHSO₄).

Compounds such as

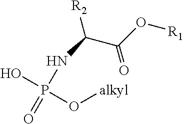

can be made by reacting alkyl phosphorodichloridate in a suitable solvent (such as DCM) in the presence of base (e.g. triethylamine) with an amino acid ester and benzyl alcohol. The benzyl group can then be removed by hydrogenolysis, over a suitable catalyst (e.g Pd(OH)₂/C).

For compounds where X is NH, Y′ is bond and Z′ is CHR₂′(C═O)OR₁′ such as

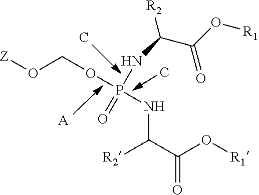

Then the principle connections required are A and C as shown.

Connection A is described above, using

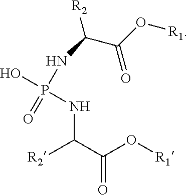

Connection C can be made to make compounds such as

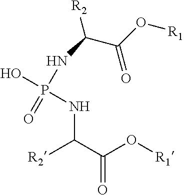

in the same manner as connection B, but using POCl₃ as a starting material instead of an alkyl phosphorodichloridate.

For compounds where X′ is O, Y′ is bond and Z′ is alkyl and Y is PhCH₂O such as

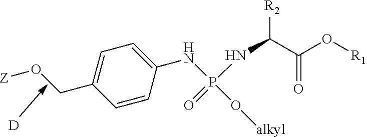

Then the principle connection is D as shown. This can be made via a nucleophilic displacement of a hydroxyl group by a method in which a compound as shown above where Z is H is reacted with a suitable phenol (e.g. DNP or salicylanilide) in the presence of activating reagents (typically DIAD and PPh₃) in a suitable solvent such as THF. The compound where Z is H can be made by methods including reacting a made by reacting an alkyl phosphorodichloridate in a suitable solvent (such as DCM) in the presence of base (e.g. triethylamine) with an amino acid ester, O-protected aniline and benzyl alcohol. The benzyl group can then be removed by hydrogenolysis, over a suitable catalyst (e.g Pd(OH)2/C). The protection group of the aniline (typically TBS) can then be removed by the action of, for instance, TBAF in a suitable solvent, e.g. THF.

Compounds such as

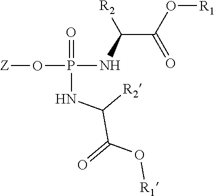

can be made by reacting POCl₃ with an amino acid ester and a suitable phenol (such as salicylanilide) in the presence of a base (typically triethylamine) in a non-nucleophilic solvent such as DCM.

Compounds such as

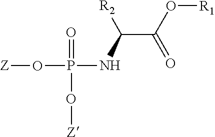

can be made by reacting POCl₃ with an amino acid ester and a suitable phenol (such as salicylanilide) in the presence of a base (typically triethylamine) in a non-nucleophilic solvent such as DCM.

Protecting groups include but are not limited to benzyl and tert-butyl. Other protecting groups for carbonyls and their removal are detailed in ‘Greene's Protective Groups in Organic Synthesis’ (Wuts and Greene, Wiley, 2006). Protecting groups may be removed by methods known to one skilled in the art including hydrogenation in the presence of a heterogenous catalyst for benzyl esters and treatment with organic or mineral acids, preferably trifluoroacetic acid or dilute HCl, for tert-butyl esters.

Where mixtures are formed then the compounds of the invention may need to be separated. One method for separating the compounds is column chromatography.

### Pharmaceutical Compositions Comprising a Compound of the Invention

The present invention also provides a pharmaceutical composition comprising the compound of the invention together with one or more pharmaceutically acceptable diluents or carriers.

The compound of the invention or a formulation thereof may be administered by any conventional route for example, but not limited to, orally, parenterally, topically, via a mucosa such as buccal, sublingual, transdermal, vaginal, rectal, nasal, ocular or, via a medical device (e.g. a stent), by inhalation or via injection (subcutaneous or intramuscular). The treatment may consist of a single dose or a plurality of doses over a period of time.

The treatment may be by administration once daily, twice daily, three times daily, four times daily etc. The treatment may also be by continuous administration such as e.g. administration intravenous by infusion (drop).

Whilst it is possible for the compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Examples of suitable carriers are described in more detail below.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general, the formulations are prepared by uniformly and intimately bringing into association the active ingredient with liquid carriers or finely divided solid carriers or both, and then, if necessary, shaping the product.

The compound of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses and/or frequencies.

The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof, or it may be a solid material eg for manufacturing of solid dosage forms.

For example, the compound of the invention can also be administered orally, buccally or sublingually in the form of tablets, capsules, ovules, elixirs, solutions or suspensions, which may contain flavouring or colouring agents, for immediate-, delayed- or controlled-release applications.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Solutions, emulsions or suspensions of the compound of the invention suitable for oral administration may also contain one or more solvents including water, alcohol, polyol etc. as well as one or more excipients such as pH-adjusting agent, stabilizing agents, surfactants, solubilizers, dispersing agents, preservatives, flavors etc. Specific examples include excipients e.g. N,N-dimethylacetamide, dispersants e.g. polysorbate 80, surfactants, and solubilisers, e.g. polyethylene glycol, Phosal 50 PG (which consists of phosphatidylcholine, soya-fatty acids, ethanol, mono/diglycerides, propylene glycol and ascorbyl palmitate). The formulations according to present invention may also be in the form of emulsions, wherein a compound according to Formula (I) may be present in an aqueous oil emulsion. The oil may be any oil-like substance such as e.g. soy bean oil or safflower oil, medium chain triglycieride (MCT-oil) such as e.g. coconut oil, palm oil etc or combinations thereof.

Tablets may contain excipients such as microcrystalline cellulose, lactose (e.g. lactose monohydrate or lactose anhydrous), sodium citrate, calcium carbonate, dibasic calcium phosphate and glycine, butylated hydroxytoluene (E321), crospovidone, hypromellose, disintegrants such as starch (preferably corn, potato or tapioca starch), sodium starch glycollate, croscarmellose sodium, and certain complex silicates, and granulation binders such as polyvinylpyrrolidone, hydroxypropylmethylcellulose (HPMC), hydroxypropylcellulose (HPC), macrogol 8000, sucrose, gelatin and acacia. Additionally, lubricating agents such as magnesium stearate, stearic acid, glyceryl behenate and talc may be included.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Formulations suitable for administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Pharmaceutical compositions adapted for topical administration may be formulated as ointments, creams, emulsions, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either colloidal, suspended or dissolved in the vehicle. In preparing solutions the active ingredient can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

It should be understood that in addition to the ingredients particularly mentioned above the formulations of this invention may include other agents conventional in the art having regard to the type of formulation in question, for example those suitable for oral administration may include flavouring agents. A person skilled in the art will know how to choose a suitable formulation and how to prepare it (see eg Remington's Pharmaceutical Sciences 18 Ed. or later). A person skilled in the art will also know how to choose a suitable administration route and dosage.

The compositions may contain from 0.1% by weight, from 5-60%, or from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

All % values mentioned herein are % w/w unless the context requires otherwise.

Any combination of such a drug substance with any compound of the invention is within the scope of the present invention. Accordingly, based on the disclosure herein a person skilled in the art will understand that the gist of the invention is the findings of the valuable properties of compounds of the invention to avoid or reduce the side-effects described herein. Thus, the potential use of compounds of the invention capable of entering cells and deliver a metabolite and possibly other active moieties in combination with any drug substance that has or potentially have the side-effects described herein is evident from the present disclosure.

### Definitions

The articles “a” and “an” are used herein to refer to one or to more than one (i.e. at least one) of the grammatical objects of the article. By way of example “an analogue” means one analogue or more than one analogue.

As used herein the term “compound(s) of the invention”, “refers to compounds of formula (I) or salicylanilide.

As used herein the term “salicylanilide” refers to a compound with the structure in formula (II):

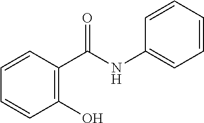 [SMILES: O=C(Nc1ccccc1)c1ccccc1O]

As used herein, the term “bioavailability” refers to the degree to which or rate at which a drug or other substance is absorbed or becomes available at the site of biological activity after administration. This property is dependent upon a number of factors including the solubility of the compound, rate of absorption in the gut, the extent of protein binding and metabolism etc. Various tests for bioavailability that would be familiar to a person of skill in the art are described herein (see also Trepanier et al, 1998, Gallant-Haidner et al, 2000).

The pharmaceutically acceptable salts of the compound of the invention include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

As used herein the term “alkyl” refers to any straight or branched chain composed of only sp³ carbon atoms, fully saturated with hydrogen atoms such as e.g. —CₙH₂ₙ₊₁ for straight chain alkyls, wherein n can be in the range of 1 and 10 such as e.g. methyl, ethyl, propyl, isopropyl, n-butyl, isobutyl, sec-butyl, tert-butyl, n-pentyl, neopentyl, isopentyl, hexyl, isohexyl, heptyl, octyl, nonyl or decyl. The alkyl as used herein may be further substituted.

As used herein the term “cycloalkyl” refers to a cyclic/ring structured carbon chains having the general formula of —CₙH₂ₙ₋₁ where n is between 3-10, such as e.g. cyclopropyl, cyclobutyl, cyclopentyl, cyclohexyl, cycloheptyl or cyclooctyl, bicycle[3.2.1]octyl, spiro[4,5]decyl, norpinyl, norbonyl, norcapryl, adamantly and the like. The cycloalkyl as used herein may be further substituted.

As used herein, the term “alkenyl” refers to a straight or branched chain composed of carbon and hydrogen atoms wherein at least two carbon atoms are connected by a double bond such as e.g. C₂₋₁₀ alkenyl unsaturated hydrocarbon chain having from two to ten carbon atoms and at least one double bond. C₂₋₆ alkenyl groups include, but are not limited to, vinyl, 1-propenyl, allyl, iso-propenyl, n-butenyl, n-pentenyl, n-hexenyl and the like. The alkenyl as used herein may be further substituted.

As used herein the term “cycloalkenyl” refers to a cyclic/ring structured carbon chains having the general formula of —CₙH₂ₙ₋₁ where n is between 3-10, wherein at least two carbon atoms are connected by a double bond such as e.g. cyclopropenyl, cyclobutenyl, cyclopentenyl, cyclohexenyl, cycloheptenyl, cyclooctenyl, norbornenyl or bic-clo[2.2.2]oct2enyl. The cycloalkenyl as used herein may be further substituted.

The term “C₁₋₁₀ alkoxy” in the present context designates a group —O—C-₁₋₁₀ alkyl used alone or in combination, wherein C₁₋₁₀ alkyl is as defined above. Examples of linear alkoxy groups are methoxy, ethoxy, propoxy, butoxy, pentoxy and hexoxy. Examples of branched alkoxy are iso-propoxy, sec-butoxy, tert-butoxy, iso-pentoxy and iso-hexoxy. Examples of cyclic alkoxy are cyclopropyloxy, cyclobutyloxy, cyclopentyloxy and cyclohexyloxy.

The term “C₃₋₇ heterocycloalkyl” as used herein denotes a totally saturated heterocycle like a cyclic hydrocarbon containing one or more heteroatoms selected from nitrogen, oxygen and sulfur independently in the cycle. Examples of heterocycles include, but are not limited to, pyrrolidine (1-pyrrolidine, 2-pyrrolidine, 3-pyrrolidine, 4-pyrrolidine, 5-pyrrolidine), pyrazolidine (1-pyrazolidine, 2-pyrazolidine, 3-pyrazolidine, 4-pyrazolidine, 5-pyrazolidine), imidazolidine (1-imidazolidine, 2-imidazolidine, 3-imidazolidine, 4-imidazolidine, 5-imidazolidine), thiazolidine (2-thiazolidine, 3-thiazolidine, 4-thiazolidine, 5-thiazolidine), piperidine (1-piperidine, 2-piperidine, 3-piperidine, 4-piperidine, 5-piperidine, 6-piperidine), piperazine (1-piperazine, 2-piperazine, 3-piperazine, 4-piperazine, 5-piperazine, 6-piperazine), morpholine (2-morpholine, 3-morpholine, 4-morpholine, 5-morpholine, 6-morpholine), thiomorpholine (2-thiomorpholine, 3-thiomorpholine, 4-thiomorpholine, 5-thiomorpholine, 6-thiomorpholine), 1,2-oxathiolane (3-(1,2-oxathiolane), 4-(1,2-oxathiolane), 5-(1,2-oxathiolane)), 1,3-dioxolane (2-(1,3-dioxolane), 3-(1,3-dioxolane), 4-(1,3-dioxolane)), tetrahydropyrane (2-tetrahydropyrane, 3-tetrahydropyrane, 4-tetrahydropyrane, 5-tetrahydropyrane, 6-tetrahydropyrane), hexahydropyradizine, (1 (hexahydropyradizine), 2-(hexahydropyradizine), 3-(hexahydropyradizine), 4-(hexahydropyradizine), 5-(hexahydropyradizine), 6-(hexahydropyradizine)).

The term “C₁₋₁₀alkyl-C₃₋₁₀cycloalkyl” as used herein refers to a cycloalkyl group as defined above attached through an alkyl group as defined above having the indicated number of carbon atoms.

The term “aryl” as used herein is intended to include carbocyclic aromatic ring systems.

Aryl is also intended to include the partially hydrogenated derivatives of the carbocyclic systems enumerated below.

The term “heteroaryl” as used herein includes heterocyclic unsaturated ring systems containing one or more heteroatoms selected among nitrogen, oxygen and sulfur, such as furyl, thienyl, pyrrolyl, and is also intended to include the partially hydrogenated derivatives of the heterocyclic systems enumerated below.

The terms “aryl” and “heteroaryl” as used herein refers to an aryl, which can be optionally unsubstituted or mono-, di- or tri substituted, or a heteroaryl, which can be optionally unsubstituted or mono-, di- or tri substituted. Examples of “aryl” and “heteroaryl” include, but are not limited to, phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), phenanthrenyl, fluorenyl, pentalenyl, azulenyl, biphenylenyl, thiophenyl (1-thienyl, 2-thienyl), furyl (1-furyl, 2-furyl), furanyl, thiophenyl, isoxazolyl, isothiazolyl, 1,2,3-triazolyl, 1,2,4-triazolyl, pyranyl, pyridazinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indolyl, oxadiazolyl, isoxazolyl, quinazolinyl, fluorenyl, xanthenyl, isoindanyl, benzhydryl, acridinyl, benzisoxazolyl, purinyl, quinazolinyl, quinolizinyl, quinolinyl, isoquinolinyl, quinoxalinyl, naphthyridinyl, phteridinyl, azepinyl, diazepinyl, pyrrolyl (2-pyrrolyl), pyrazolyl (3-pyrazolyl), 5-thiophene-2-yl-2H-pyrazol-3-yl, imidazolyl (1-imidazolyl, 2-imidazolyl, 4-imidazolyl, 5-imidazolyl), triazolyl (1,2,3-triazol-1-yl, 1,2,3-triazol-2-yl, 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), isoquinolyl (1-isoquinolyl, 3-isoquinolyl, 4-isoquinolyl, 5-isoquinolyl, 6-isoquinolyl, 7-isoquinolyl, 8-isoquinolyl), quinolyl (2-quinolyl, 3-quinolyl, 4-quinolyl, 5-quinolyl, 6-quinolyl, 7-quinolyl, 8-quinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl)), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), indolyl (1-indolyl, 2-indolyl, 3-indolyl, 4-indolyl, 5-indolyl, 6-indolyl, 7-indolyl), indazolyl (1-indazolyl, 2-indazolyl, 3-indazolyl, 4-indazolyl, 5-indazolyl, 6-indazolyl, 7-indazolyl), benzimidazolyl, (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl). Non-limiting examples of partially hydrogenated derivatives are 1,2,3,4-tetrahydronaphthyl, 1,4-dihydronaphthyl, pyrrolinyl, pyrazolinyl, indolinyl, oxazolidinyl, oxazolinyl, oxazepinyl and the like.

As used herein the term “acyl” refers to a carbonyl group —C(═O) R wherein the R group is any of the above defined groups. Specific examples are formyl, acetyl, propionyl, butyryl, pentanoyl, hexanoyl, heptanoyl, octanoyl, nonanoyl, decanoyl, benzoyl and the likes.

“Optionally substituted” as applied to any group means that the said group may, if desired, be substituted with one or more substituents, which may be the same or different. ‘Optionally substituted alkyl’ includes both ‘alkyl’ and ‘substituted alkyl’.

Examples of suitable substituents for “substituted” and “optionally substituted” moieties include halo (fluoro, chloro, bromo or iodo), C₁₋₆ alkyl, C₃₋₆ cycloalkyl, C₃₋₆ cycloalkenyl hydroxy, C₁₋₆ alkoxy, cyano, amino, nitro, C₁₋₆ alkylamino, C₂₋₆ alkenylamino, di-C₁₋₆ alkylamino, C₁₋₆ acylamino, di-C₁₋₆ acylamino, C₁₋₆ aryl, C₁₋₆ arylamino, C₁₋₆ aroylamino, benzylamino, C₁₋₆ arylamido, carboxy, C₁₋₆ alkoxycarbonyl or (C₁₋₆ aryl)(C₁₋₁₀ alkoxy)carbonyl, carbamoyl, mono-C₁₋₆ carbamoyl, di-C₁₋₆ carbamoyl or any of the above in which a hydrocarbyl moiety is itself substituted by halo, cyano, hydroxy, C₁₋₂ alkoxy, amino, nitro, carbamoyl, carboxy or C₁₋₂ alkoxycarbonyl. In groups containing an oxygen atom such as hydroxy and alkoxy, the oxygen atom can be replaced with sulfur to make groups such as thio (SH) and thio-alkyl (S-alkyl). Optional substituents therefore include groups such as S-methyl. In thio-alkyl groups, the sulfur atom may be further oxidised to make a sulfoxide or sulfone, and thus optional substituents therefore includes groups such as S(O)-alkyl and S(O)₂-alkyl.

Substitution may take the form of double bonds, and may include heteroatoms. Thus an alkyl group with a carbonyl (C═O) instead of a CH₂ can be considered a substituted alkyl group.

Substituted groups thus include for example CFH₂, CF₂H, CF₃, CH₂NH₂, CH₂OH, CH₂CN, CH₂SCH₃, CH₂OCH₃, OMe, OEt, Me, Et, —OCH₂O—, CO₂Me, C(O)Me, i-Pr, SCF₃, SO₂Me, NMe₂, CONH₂, CONMe₂ etc. In the case of aryl groups, the substitutions may be in the form of rings from adjacent carbon atoms in the aryl ring, for example cyclic acetals such as O—CH₂—O.

### LEGENDS TO FIGURES

FIG. 1 show the results of free mitochondrial uncoupling of compounds 1 and 2 compared with known potent uncoupler DNP

FIG. 2 show the results of free mitochondrial uncoupling of compound 4 compared with known potent uncoupler MNP

FIG. 3 shows the result of salicylanilide and DNP in a mitochondrial uncoupling assay in intact HepG2 liver cells.

FIG. 4A shows the result of compound 11 in an isolated mitochondrial uncoupling assay, compared to a DMSO negative control and DNP positive control.

FIG. 4B shows the result of compound 9 in an isolated mitochondrial uncoupling assay, compared to a DMSO negative control and MNP positive control.

FIG. 5A shows the result of compound 6 in an isolated mitochondrial uncoupling assay, compared to a DMSO negative control and Niclosamide control.

FIG. 5B shows the result of compound 18 in an isolated mitochondrial uncoupling assay, compared to a DMSO negative control and Niclosamide control.

FIG. 6A shows the result of compound 23 in an isolated mitochondrial uncoupling assay, compared to a DMSO negative control and salicylanilide control.

FIG. 6B shows the result of compound 14 in an isolated mitochondrial uncoupling assay, compared to a DMSO negative control and salicylanilide control.

FIG. 7 shows the result of compound 11 in a mitochondrial uncoupling assay in intact HepG2 liver cells, platelets in comparison to DMSO negative control and DNP.

FIG. 8 shows the result of compound 9 in a mitochondrial uncoupling assay in intact HepG2 liver cells, platelets in comparison to DMSO negative control and MNP.

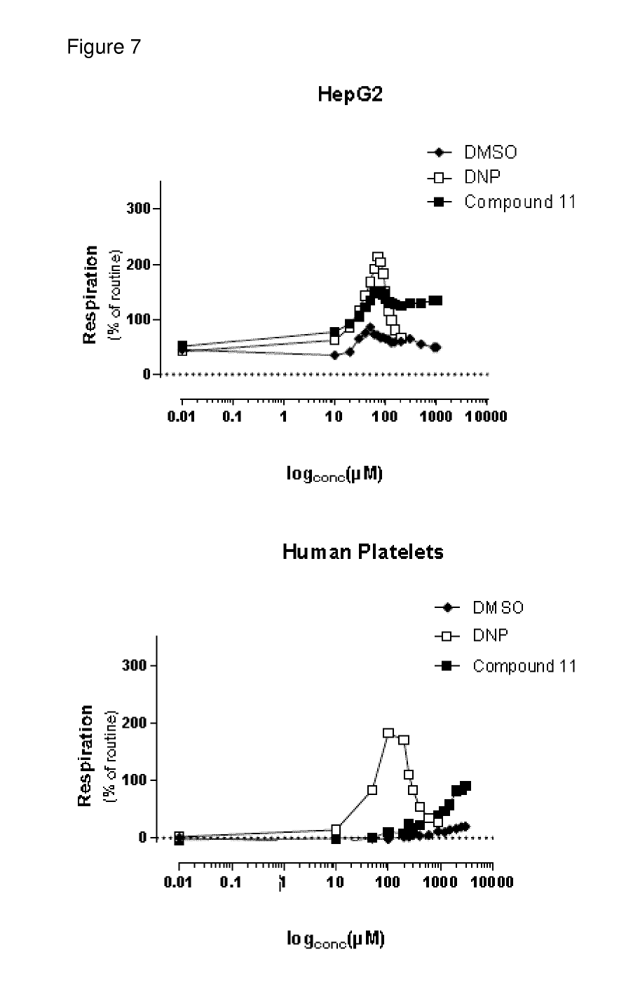

FIG. 7 shows the result of compound 11 in a mitochondrial uncoupling assay in intact HepG2 liver cells, platelets in comparison to DMSO negative control and DNP.

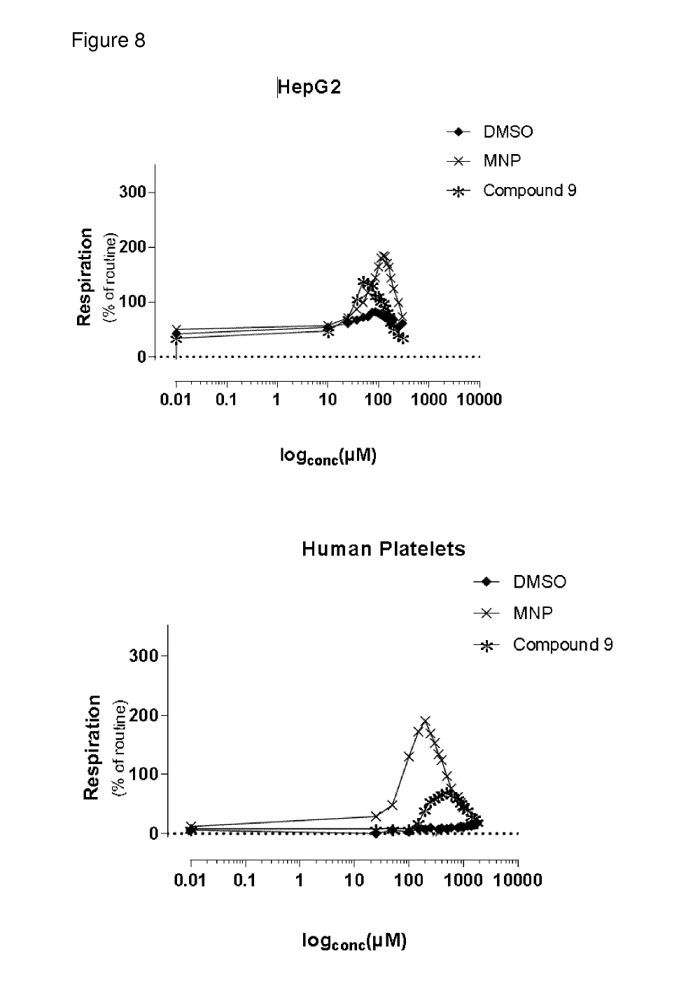

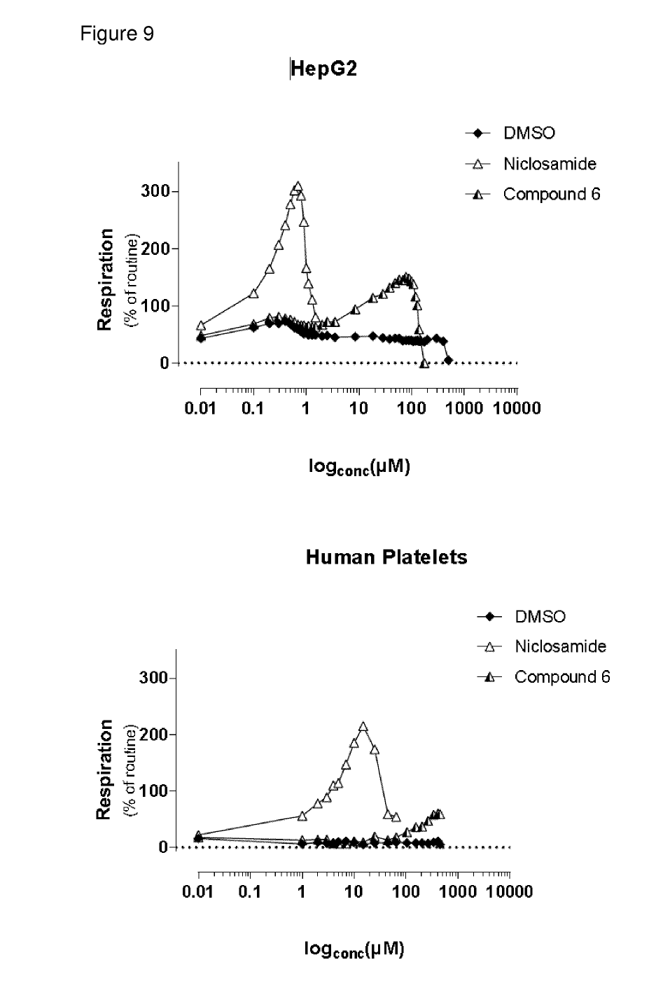

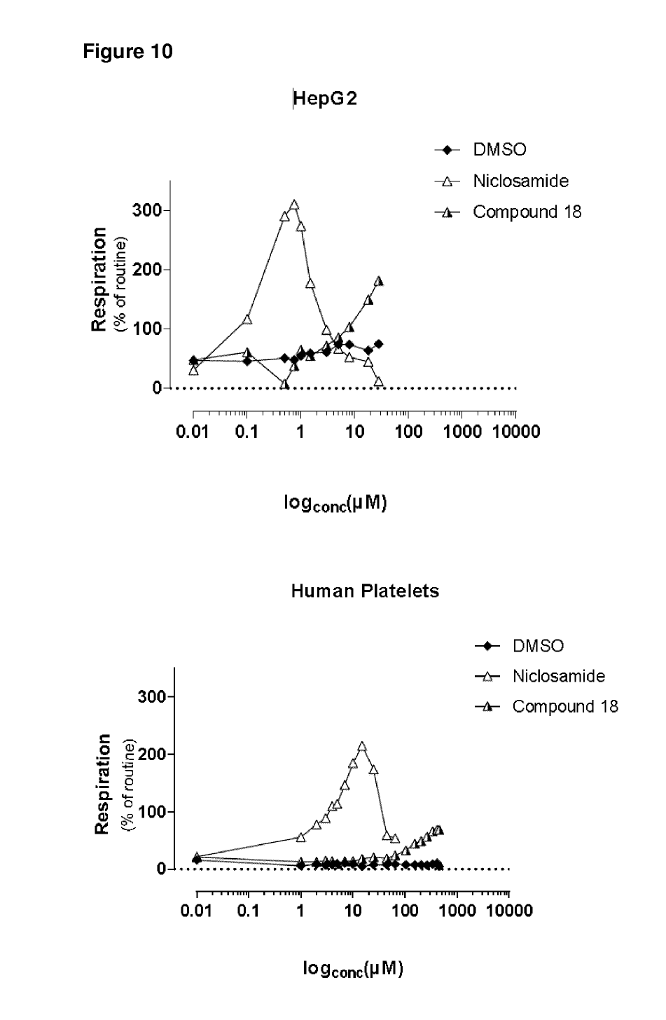

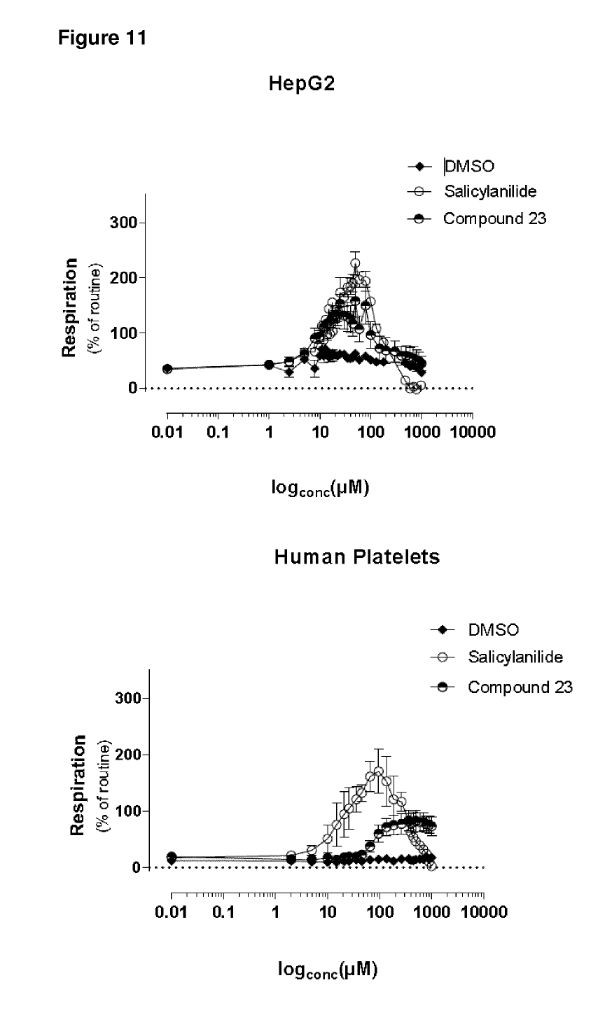

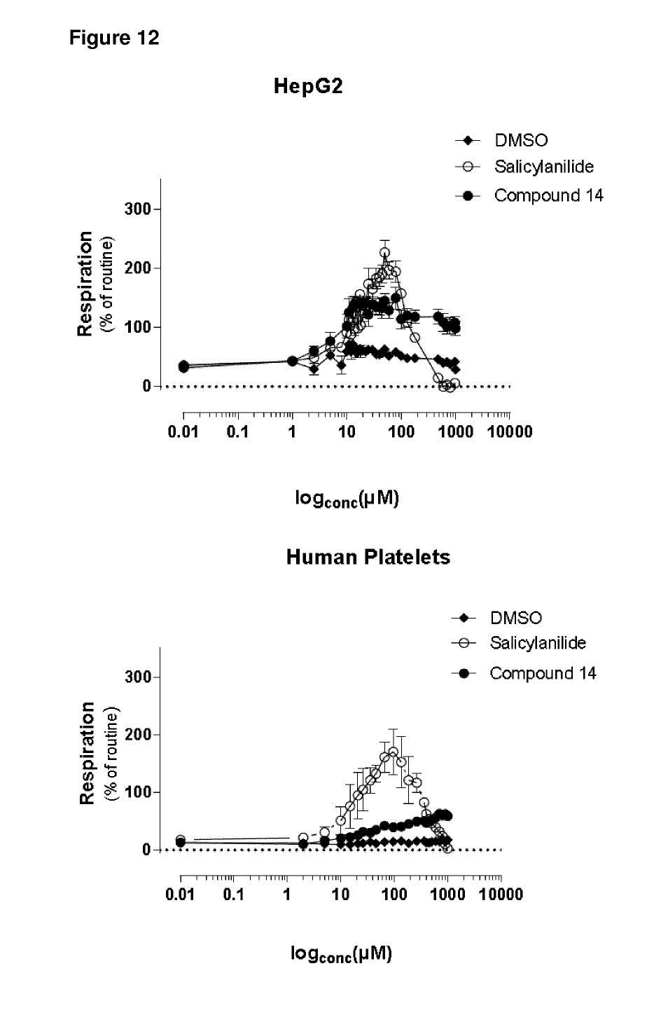

FIG. 8 shows the result of compound 9 in a mitochondrial uncoupling assay in intact HepG2 liver cells, platelets in comparison to DMSO negative control and MNP.

FIG. 9 shows the result of compound 6 in a mitochondrial uncoupling assay in intact HepG2 liver cells, platelets in comparison to DMSO negative control and Niclosamide.

FIG. 10 shows the result of compound 18 in a mitochondrial uncoupling assay in intact HepG2 liver cells, platelets in comparison to DMSO negative control and Niclosamide.

FIG. 11 shows the result of compound 23 in a mitochondrial uncoupling assay in intact HepG2 liver cells, platelets in comparison to DMSO negative control and salicylanilide.

FIG. 12 shows the result of compound 14 in a mitochondrial uncoupling assay in intact HepG2 liver cells, platelets in comparison to DMSO negative control and salicylanilide.

### EXPERIMENTAL

A broad series of protonophore chemical classes were assessed for mitochondrial uncoupling activity to look for uncoupling potency in combination with low cellular toxicity. Liver-targeted prodrugs were then generated and tested in preclinical models.

Assessment of mitochondrial uncoupling activity revealed a number of classes of protonophores, which showed significantly less toxicity than DNP, but with improved uncoupling potency. A series of prodrugs were then generated with chemistry aimed to liver-target the protonophore. The prodrugs induced uncoupled mitochondrial respiration in liver cells with low micromolar potencies similar to the payload protonophores but lacked effect on isolated liver mitochondria. The therapeutic range of respiratory stimulation was widened and the maximal induced respiration was less than half compared to the payload protonophores. Compounds of the invention will be selected and investigated for impact on a number of preclinical markers of NASH and tolerability. Preclinical assessment of compounds of the invention suggests that it can cause liver-targeted mild mitochondrial uncoupling, without off-target issues associated with historical mitochondrial uncouplers, such as DNP. Preclinical assessment suggests it has potential as a treatment for NAFLD and NASH.

### General Biology Methods

### Measurement of Bioavailability

A person of skill in the art will be able to determine the pharmacokinetics and bioavailability of the compound of the invention using in vivo and in vitro methods known to a person of skill in the art, including but not limited to those described below and in Gallant-Haidner et al, 2000 and Trepanier et al, 1998 and references therein. This can be used to determine the relative exposure of the protonophore moiety in liver versus muscle and other organs. The bioavailability of a compound is determined by a number of factors, (e.g. water solubility, cell membrane permeability, the extent of protein binding and metabolism and stability) each of which may be determined by in vitro tests as described in the examples herein, it will be appreciated by a person of skill in the art that an improvement in one or more of these factors will lead to an improvement in the bioavailability of a compound. Alternatively, the bioavailability of the compound of the invention may be measured using in vivo methods as described in more detail below, or in the examples herein.

In order to measure bioavailability in vivo, a compound may be administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below.

For example, mice or rats are dosed with 1 or 3 mg/kg of the compound of the invention i.v. or 1, 5 or 10 mg/kg of the compound of the invention p.o. Blood samples are taken at 5 min, 15 min, 1 h, 4 h and 24 h intervals, and the concentration of the compound of the invention in the sample is determined via LCMS-MS. The time-course of plasma or whole blood concentrations can then be used to derive key parameters such as the area under the plasma or blood concentration-time curve (AUC—which is directly proportional to the total amount of unchanged drug that reaches the systemic circulation), the maximum (peak) plasma or blood drug concentration, the time at which maximum plasma or blood drug concentration occurs (peak time), additional factors which are used in the accurate determination of bioavailability include: the compound's terminal half-life, total body clearance, steady-state volume of distribution and F %.

These parameters are then analysed by non-compartmental or compartmental methods to give a calculated percentage bioavailability, for an example of this type of method see Gallant-Haidner et al, 2000 and Trepanier et al, 1998, and references therein.

### Efficacy Measurement

The efficacy of the compound of the invention may be tested using one or more of the methods described below:

### 1. Assays for Evaluating Mitochondrial Uncoupling

### Assay for Evaluating Uncoupling Potential in Isolated Mitochondria

The potency of mitochondrial uncoupling without prodrug metabolism may be tested as follows:

Isolated rat liver mitochondria are prepared according to Hansson et al (Hansson et al (Brain Res. 2003 Jan. 17; 960(1-2):99-111.). Respiration is measured at a constant temperature of 37° C. in a high-resolution oxygraph (Oxygraph-2k Oroboros Instruments, Innsbruck, Austria) in 2 ml glass chambers with stirrer speed 750 rpm. Data is recorded with DatLab software (Oroboros Instruments, Innsbruck, Austria) with sampling rate set to 2 s at an oxygen concentration in the range of 210-50 μM O₂. If necessary, reoxygenation is performed by partially raising the chamber stopper for a brief air equilibration. Instrumental background oxygen flux is measured in a separate set of experiments and automatically corrected for in the ensuing experiments according to the manufacturer's instructions. To measure respiration of isolated mitochondria, samples are suspended in a mitochondrial respiration medium (MiR05) containing sucrose 110 mM, HEPES 20 mM, taurine 20 mM, K-lactobionate 60 mM, MgCl₂ 3 mM, KH₂PO₄ 10 mM, EGTA 0.5 mM, BSA 1 g/l, pH 7.1. After reaching stabilized respiration in the presence of substrates (malate (5 mM), glutamate (5 mM), pyruvate (5 mM) and succinate (10 mM)), state 3 respiration is induced by supplementation with ADP (1 mM) followed by addition of oligomycin (1 μg/ml, ATP-synthase inhibitor) causing state 4_{O}. State 4_{O} is a respiratory state dependent on the back-flux of protons across the mitochondrial membrane due to inhibition of the ATP-synthase and in the presence of saturating substrate concentrations and ADP. Drug candidates and their respective payloads of known protonophores are given at fixed concentrations to induce uncoupled respiration. Rotenone (2 μM, complex I [CI] inhibitor), antimycin-A (1 μg/ml, complex III [CIII] inhibitor) and sodium azide (10 mM) are then added to inhibit the ETS providing the residual, non-mitochondrial oxygen consumption which all respiratory values are corrected for.

### 2. Assays for Evaluating Mitochondrial Uncoupling in Intact Liver Cells and Platelets

For respiration measurements in HepG2 cells and platelets, cells are suspended in a mitochondrial respiration medium MiR05 at 37° C. in a high-resolution oxygraph (Oxygraph-2k Oroboros Instruments, Innsbruck, Austria). Initially, samples are left to stabilise at a routine respiration state, revealing resting cellular energy demands on oxidative phosphorylation (OXPHOS) of endogenous substrates. To evaluate the contribution of respiration independent of ADP phosphorylation, oligomycin (1 μg/ml, ATP-synthase inhibitor) is sequentially added inducing LEAK respiration state (a respiratory state where oxygen consumption is dependent on the back-flux of protons across the mitochondrial membrane). Drug candidates and known protonophores are carefully titrated to induce maximal uncoupled respiration/maximal rate of the ETS (electron transport system) at endogenous substrate supply and continued until a decrease or at least no further increase of uncoupled respiration is observed. Rotenone (2 μM, complex I [CI] inhibitor) and antimycin-A (1 μg/ml, complex III [CIII] inhibitor) are then added to inhibit the ETS, thus providing the residual, non-mitochondrial oxygen consumption, which all values were corrected for.

The potency of mitochondrial uncoupling with prodrug metabolism be tested as follows:
a) HepG2 cells (to simulate uncoupling with liver cell metabolism)
b) Platelets (to simulate uncoupling in blood)

### Hepatocyte Stability Assay

Cryopreserved hepatocytes, previously stored in liquid nitrogen are placed in a 37±1° C. shaking water bath for 2 min±15 sec. The hepatocytes are then added to 1 OX volume of pre-warmed Krebs-Henseleit bicarbonate (KHB) buffer (2000 mg/L glucose, without calcium carbonate and sodium bicarbonate, Sigma), mixed gently and centrifuged at 500 rpm for 3 minutes. After centrifugation, the supernatant is carefully removed and a 10× volume of pre-warmed KHB buffer added to resuspend the cell pellet. This is mixed gently and centrifuged at 500 rpm for 3 minutes. The supernatant is then removed and discarded. The cell viability and yield are then determined by cell counts, and these values used to generate human hepatocyte suspensions to the appropriate seeding density (viable cell density=2×106 cells/mL). A 2× dosing solution is prepared in pre-warmed KHB (1% DMSO) (200 μM spiking solution: 20 μL of substrate stock solution (10 mM) in 980 μL of DMSO, 2× dosing solution: 10 μL of 200 μM spiking solution in 990 μL of KHB (2 μM after dilution). 50 μL of pre-warmed 2× dosing solution is added to the wells and 50 μL of pre-warmed hepatocyte solution (2×106 cells/mL) added and timing started. The plate is then incubated at 37° C. 100 μL of acetonitrile containing internal standard is added to each the wells after completion of incubation time (0, 15, 30, 60 and 120 minutes) mixed gently, and 50 μL of pre-warmed hepatocyte solution added (2×106 cells/mL). At the end of the incubation, cell viability is determined. Samples are centrifuged at 4000 rpm for 15 minutes at 4° C., supernatants diluted 2-fold with ultrapure water and compound levels analysed by LC-MS/MS.

Test compounds are prepared as stock solutions in DMSO at 10 mM concentration. The stock solutions are diluted in duplicate into PBS, pH7.4 in 1.5 mL Eppendorf tubes to a target concentration of 100 μM with a final DMSO concentration of 1% (e.g. 4 μL of 10 mM DMSO stock solution into 396 μL 100 mM phosphate buffer). Sample tubes are then gently shaken for 4 hours at room temperature. Samples are centrifuged (10 min, 15000 rpm) to precipitate undissolved particles. Supernatants are transferred into new tubes and diluted (the dilution factor for the individual test article is confirmed by the signal level of the compound on the applied analytical instrument) with PBS. Diluted samples are then mixed with the same volume (1:1) of MeOH. Samples are finally mixed with the same volume (1:1) of ACN containing internal standard for LC-MS/MS analysis. Apparent permeability coefficient (Papp) and efflux ratio of the compound across the monolayer are calculated as follows:

The permeability coefficient (Papp) is calculated from the following equation:

$${P}_{app} = ( \frac{dQ / dt}{{C}_{0} \times A} )$$

Where dQ/dt is the amount of compound in basal (A-B) or apical (B-A) compartment as a function of time (nmol/s). C0 is the initial concentration in the donor (apical or basal) compartment (Mean of T=0) (nmol/mL) and A is the area of the transwell (cm²).

The efflux ratio is then calculated as:

$$\frac{{P}_{app ( BtoA )}}{{P}_{app ( AtoB )}} .$$

### Water Solubility Assay

Water solubility may be tested as follows: A 10 mM stock solution of the compound is prepared in 100% DMSO at room temperature. Triplicate 0.01 mL aliquots are made up to 0.5 mL with either 0.1 M PBS, pH 7.3 solution or 100% DMSO in amber vials. The resulting 0.2 mM solutions are shaken, at room temperature on an IKA® vibrax VXR shaker for 6 h, followed by transfer of the resulting solutions or suspensions into 2 mL Eppendorf tubes and centrifugation for 30 min at 13200 rpm. Aliquots of the supernatant fluid are then analysed by the LCMS method as described above.

Alternatively, solubility in PBS at pH7.4 may be tested as follows: A calibration curve is generated by diluting the test compounds and control compounds to 40 μM, 16 μM, 4 μM, 1.6 μM, 0.4 μM, 0.16 μM, 0.04 μM and 0.002 μM, with 50% MeOH in H2O. The standard points are then further diluted 1:20 in MeOH:PBS 1:1. The final concentrations after 1:20 dilution are 2000 nM, 800 nM, 200 nM, 80 nM, 20 nM, 8 nM, 2 nM and 1 nM. Standards are then mixed with the same volume (1:1) of ACN containing internal standard. The samples are centrifuged (5 min, 12000 rpm), then analysed by LC/MS.

### Cell Permeability Assay

### Caco-2 Permeability Assay

Cell permeability may be tested as follows: The test compound is dissolved to 10 mM in DMSO and then diluted further in buffer to produce a final 10 μM dosing concentration. The fluorescence marker lucifer yellow is also included to monitor membrane integrity. Test compound is then applied to the apical (A) surface of Caco-2 cell monolayers and compound permeation into the basolateral (B) compartment is measured. This is performed in the reverse direction (basolateral to apical) to investigate active transport (efflux). LC-MS/MS is used to quantify levels of both the test and standard control compounds (such as Propanolol and Acebutolol).

### Materials

Unless otherwise indicated, all reagents used in the examples below are obtained from commercial sources.

### EXAMPLES

Compounds of the invention were characterised by a combination of NMR spectroscopy and mass spectrometry. The examples illustrate the following compounds, but the invention is not limited thereto.

Where Formula IV is

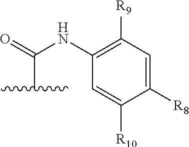

### and Formula III is

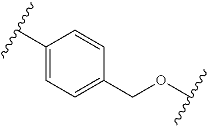 [SMILES: *OCc1ccc(*)cc1]

| compound | R1 | R2 | X | Y | Z | X′ | Y′ | Z′ |
|---|---|---|---|---|---|---|---|---|
| 1 | iPr | Me | O | —CR₃R₄O— | Formula | O | absent | Me |
| | | | | | II | | | |
| 2 | iPr | Me | O | —CR₃R₄O— | Formula | NH | absent | —CHR₂′(C═O)OR₁′ |
| | | | | | II | | | |
| 3 | iPr | Me | NH | Formula III | Formula | O | absent | Me |
| | | | | | II | | | |
| 4 | iPr | Me | O | —CR₃R₄O— | Formula | NH | absent | —CHR₂′(C═O)OR₁′ |
| | | | | | II | | | |
| 5 | iPr | Me | NH | Formula III | Formula | O | absent | Me |
| | | | | | II | | | |
| 6 | iPr | Me | O | —CR₃R₄O— | Formula | NH | absent | —CHR₂′(C═O)OR₁′ |
| | | | | | II | | | |
| 7 | iPr | Me | O | —CR₃R₄O— | Formula | NH | absent | —CHR₂′(C═O)OR₁′ |
| | | | | | II | | | |
| 8 | Me | Me | O | —CR₃R₄O— | Formula | NH | absent | —CHR₂′(C═O)OR₁′ |
| | | | | | II | | | |
| 9 | CH₂CMe₃ | Me | O | —CR₃R₄O— | Formula | NH | absent | —CHR₂′(C═O)OR₁′ |
| | | | | | II | | | |
| 10 | Et | Me | O | —CR₃R₄O— | Formula | NH | absent | —CHR₂′(C═O)OR₁′ |
| | | | | | II | | | |
| 11 | iPr | Bn | O | —CR₃R₄O— | Formula | NH | absent | —CHR₂′(C═O)OR₁′ |
| | | | | | II | | | |
| 12 | iPr | Bn | O | —CR₃R₄O— | Formula | NH | absent | —CHR₂′(C═O)OR₁′ |
| | | | | | II | | | |
| 13 | iPr | Bn | O | —CR₃R₄O— | Formula | NH | absent | —CHR₂′(C═O)OR₁′ |
| | | | | | II | | | |
| 14 | iPr | Bn | O | —CR₃R₄O— | Formula | NH | absent | —CHR₂′(C═O)OR₁′ |
| | | | | | II | | | |
| 15 | Me | Me | O | —CR₃R₄O— | Formula | O | absent | Me |
| | | | | | II | | | |
| 16 | iPr | Me | O | —CR₃R₄O— | Formula | O | absent | Et |
| | | | | | II | | | |
| 17 | iPr | Me | O | —CR₃R₄O— | Formula | O | absent | Ph |
| | | | | | II | | | |
| 18 | iPr | Me | O | —CR₃R₄O— | Formula | O | absent | Me |
| | | | | | II | | | |
| 19 | iPr | Me | O | —CR₃R₄O— | Formula | O | absent | Me |
| | | | | | II | | | |
| 20 | CH₂CMe₃ | Me | O | —CR₃R₄O— | Formula | O | absent | Me |
| | | | | | II | | | |
| 21 | Et | Me | O | —CR₃R₄O— | Formula | O | absent | Me |
| | | | | | II | | | |
| 22 | iPr | Me | O | absent | Formula | NH | absent | —CHR₂′(C═O)OR₁′ |
| | | | | | II | | | |
| 23 | CH₂CMe₃ | Me | O | —CR₃R₄O— | Formula | NH | absent | —CHR₂′(C═O)OR₁′ |
| | | | | | II | | | |
| 24 | iPr | Me | O | absent | Formula | O | absent | Formula II |
| | | | | | II | | | |
| 25 | iPr | Me | O | —CR₃R₄O— | Formula | NH | absent | —CHR₂′(C═O)OR₁′ |
| | | | | | II | | | |
| 26 | tBu | iPr | O | —CR₃R₄O— | Formula | NH | absent | —CHR₂′(C═O)OR₁′ |
| | | | | | II | | | |
| 27 | CH₂CMe₃ | Bn | O | —CR₃R₄O— | Formula | NH | absent | —CHR₂′(C═O)OR₁′ |
| | | | | | II | | | |

| compound | R1′ | R2′ | R3 | R4 | R5 | R6 | R7 | R8 | R9 | R10 |
|---|---|---|---|---|---|---|---|---|---|---|
| 1 | NA | NA | H | H | NO₂ | NO₂ | H | NA | NA | NA |
| 2 | iPr | Me | H | H | NO₂ | NO₂ | H | NA | NA | NA |
| 3 | NA | NA | NA | NA | NO₂ | NO₂ | H | NA | NA | NA |
| 4 | iPr | Me | H | H | H | NO₂ | H | NA | NA | NA |
| 5 | NA | NA | NA | NA | H | NO₂ | H | NA | NA | NA |
| 6 | iPr | Me | H | H | Formula IV | Cl | H | NO₂ | Cl | H |
| 7 | iPr | Me | H | H | Formula IV | H | H | H | H | H |
| 8 | Me | Me | H | H | H | NO₂ | H | NA | NA | NA |
| 9 | CH₂CMe₃ | Me | H | H | H | NO₂ | H | NA | NA | NA |
| 10 | Et | Me | H | H | H | NO₂ | H | NA | NA | NA |
| 11 | iPr | Bn | H | H | NO₂ | NO₂ | H | NA | NA | NA |
| 12 | iPr | Bn | H | H | H | NO₂ | H | NA | NA | NA |
| 13 | iPr | Bn | H | H | Formula IV | Cl | H | NO2 | Cl | H |
| 14 | iPr | Bn | H | H | Formula IV | H | H | H | H | H |
| 15 | NA | NA | H | H | H | NO₂ | H | NA | NA | NA |
| 16 | NA | NA | H | H | H | NO₂ | H | NA | NA | NA |
| 17 | NA | NA | H | H | H | NO₂ | H | NA | NA | NA |
| 18 | NA | NA | H | H | Formula IV | Cl | H | NO2 | Cl | H |
| 19 | NA | NA | H | H | Formula IV | H | H | H | H | H |
| 20 | NA | NA | H | H | H | NO₂ | H | NA | NA | NA |
| 21 | NA | NA | H | H | H | NO₂ | H | NA | NA | NA |
| 22 | iPr | Me | NA | NA | Formula IV | H | H | H | H | H |
| 23 | CH₂CMe₃ | Me | H | H | Formula IV | H | H | H | H | H |
| 24 | NA | NA | NA | NA | Formula IV | H | H | H | H | H |
| 25 | iPr | Me | H | H | Formula IV | H | H | H | H | H |
| 26 | tBu | iPr | H | H | Formula IV | H | H | H | H | H |
| 27 | CH₂CMe₃ | Bn | H | H | Formula IV | H | H | H | H | H |

### Example 1—Compound 1

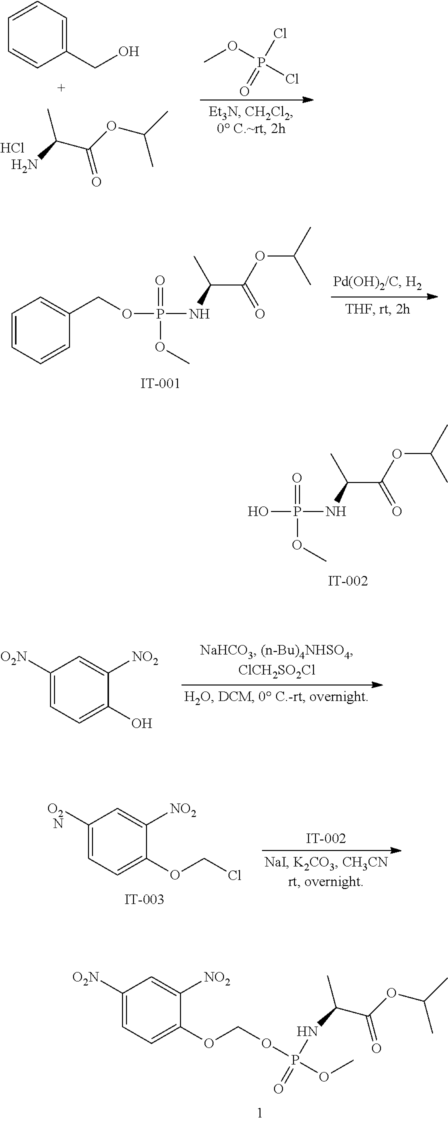

A solution of methyl phosphorodichloridate (3.0 g, 20.1 mmol) in DCM (60 ml) was added dropwise to a mixture of benzyl alcohol (2.18 g, 20.1 mmol) and triethylamine (TEA) (2.04 g, 20.1 mmol) at 0° C. under nitrogen. After addition, the reaction was stirred at room temperature for 30 min before it was re-cooled to 0° C. L-Alanine isopropyl ester hydrochloride (3.71 g, 22.2 mmol) was added to the reaction and then TEA (6.12 g, 60.4 mmol) was added dropwise. The reaction mixture was stirred at room temperature for 2 hours before it was quenched with water. The resulting mixture was extracted with DCM twice, then the combined organic layers were dried over Na₂SO₄, filtered and the solvent was removed in vacuo. The residue was purified by silica gel column chromatography to give IT-001 as a colourless oil. A mixture of IT-001 and Pd(OH)₂/C (100 mg) in THF (30 mL) was stirred at room temperature under hydrogen atmosphere (balloon) for 2 hours. The reaction mixture was filtered and then the solvent was removed in vacuo to give IT-002 as a colourless oil. Chloromethyl chlorosulfate (7.2 g, 43.4 mmol) was added to a mixture of 2,4-dinitrophenol (4.0 g, 21.7 mmol), tetrabutylammonium hydrogen sulfate (738 mg, 2.17 mmol) and NaHCO₃ (9.2 g, 109 mmol) in DCM (80 mL) and water (80 mL) at 0° C. After addition, the mixture was stirred at room temperature overnight. The mixture was diluted with water and extracted with DCM twice. The combined organic layers were dried over Na₂SO₄, filtered and then the solvent was removed in vacuo to give IT-003 as yellow oil which was used in next step without purification. A mixture of IT-002 (5.0 g, 22.2 mmol), IT-003 (4.2 g, 18.1 mmol), K₂CO₃ (3.75 g, 27.2 mmol) and NaI (543 mg, 3.62 mmol) in CH₃CN (80 mL) was stirred at room temperature overnight. The mixture was filtered and washed with CH₃CN. The solvent was removed in vacuo and the residue was purified by silica gel column chromatography and then preparative-HPLC (CH₃CN/H₂O) to give the title compound as slightly yellow solid.

### Example 2—Compound 2

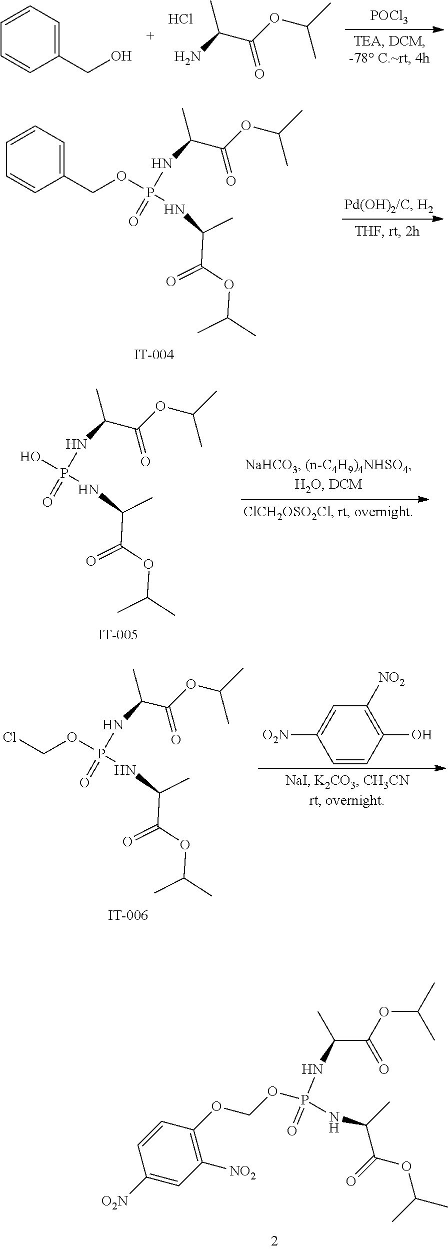

A mixture of benzylalcohol (3.39 g, 31.3 mmol) and TEA (3.96 g, 39.1 mmol) was added dropwise to a solution of phosphoryl trichloride (6.0 g, 39.1 mmol) in DCM (150 mL) at −78° C. under Ar. The mixture was stirred at −78° C. for 30 min. L-alanine isopropyl ester hydrochloride (16.4 g, 97.8 mmol) was added and then TEA (19.8 g, 196 mmol) was added dropwise into the reaction mixture. After addition, the mixture was warmed to room temperature and stirred for 4 hours. The reaction mixture was quenched with water and extracted with DCM twice. The combined organic layers were dried (Na₂SO₄), filtered and then the solvent was removed in vacuo. The residue was purified by silica gel column chromatography (EtOAc/petrol ether) to give IT-004 as colourless oil. A mixture of IT-004 (5.0 g, 12.1 mmol) and Pd(OH)₂/C (1.0 g) in THF (100 mL) was stirred at room temperature under hydrogen atmosphere (balloon) for 2 hours. The reaction mixture was filtered and evaporated under reduced pressure to give IT-005 which was used for next step without purification. Chloromethyl chlorosulfate (4.0 g, 24 mmol) was added to a mixture of IT-005 (3.9 g, 12 mmol), tetrabutylammonium hydrogen sulfate (407 mg, 1.2 mmol) and NaHCO₃ (6.0 g, 72 mmol) in DCM (60 mL) and water (60 mL) at room temperature and was stirred overnight. The mixture was extracted with DCM 3 times. The combined organic layers were dried over Na₂SO₄, filtered and then the solvent was removed in vacuo to give IT-006 as slightly yellow oil which was used in next step without purification. A mixture of IT-006 (1.8 g, 4.83 mmol), 2,4-dinitrophenol (1.33 g, 7.24 mmol), K₂CO₃ (1.34 g, 9.66 mmol) and NaI (145 mg, 0.97 mmol) in CH₃CN (27 mL) was stirred at room temperature overnight. The mixture was filtered and washed with CH₃CN. The solvent was removed in vacuo and the residue was purified by silica gel column chromatography and then preparative-HPLC (CH₃CN/H₂O) to give the title compound as colourless oil.

### Example 3—Compound 3

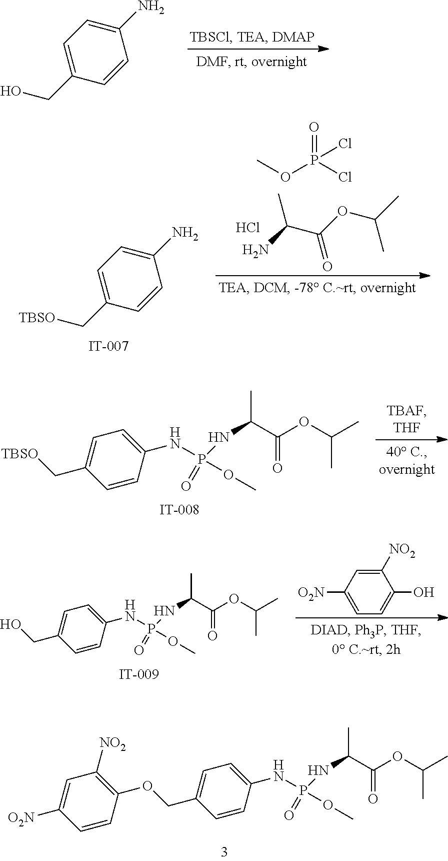

Tert-butyldimethylsilyl chloride (2.02 g, 13.4 mmol) was added to a solution of (4-aminophenyl)methanol (1.5 g, 12.2 mmol), DMAP (491 mg, 4.02 mmol) and TEA (1.48 g, 14.6 mmol) in DMF (15 mL) at room temperature and stirred at overnight. The mixture was diluted with water and extracted with EtOAc twice. The combined organic layers were washed with brine, dried (Na₂SO₄), filtered and then the solvent was removed in vacuo. The residue was purified by silica gel column chromatography (EtOAc/petrol ether) to give IT-007 as light yellow oil. A solution of IT-007 (2.3 g, 9.7 mmol) and TEA (982 mg, 9.7 mmol) in DCM (5 mL) was added dropwise to a solution of methyl phosphorodichloridate (1.44 g, 9.7 mmol) in DCM (20 mL) at −78° C. under Ar. The mixture was stirred at −78° C. for 30 min before L-alanine isopropyl ester hydrochloride (1.63 g, 9.7 mmol) was added. TEA (2.45 g, 24.3 mmol) was then added dropwise into the reaction mixture. After addition, the mixture was warmed to room temperature and stirred overnight. The reaction was quenched with water and extracted with DCM twice. The combined organic layers were dried (Na₂SO₄), filtered and then the solvent was removed in vacuo. The residue was purified by silica gel column chromatography (EtOAc/petrol ether) to give IT-008 as a colourless oil. TBAF (1 M in THF, 6.5 mL, 6.5 mmol) was added to a solution of IT-008 (970 mg, 2.18 mmol) in THF (10 mL). The reaction was heated to 40° C. and stirred overnight then the solvent was removed in vacuo. The residue was purified by silica gel column chromatography (EtOAc/petrol ether) to give IT-009 as colourless oil. DIAD (245 mg, 1.21 mmol) was added dropwise to a solution of IT-009 (100 mg, 0.303 mmol), 2,4-dinitrophenol (111 mg, 0.606 mmol) and Ph₃P (159 mg, 0.606 mmol) in THF (5 mL) at 0° C. After addition, the mixture was stirred at room temperature for 2 hours. The solvent was removed in vacuo and the residue was directly purified by preparative-TLC (EtOAc) to give the title compound as a slightly yellow solid.

### Example 4—Compound 4

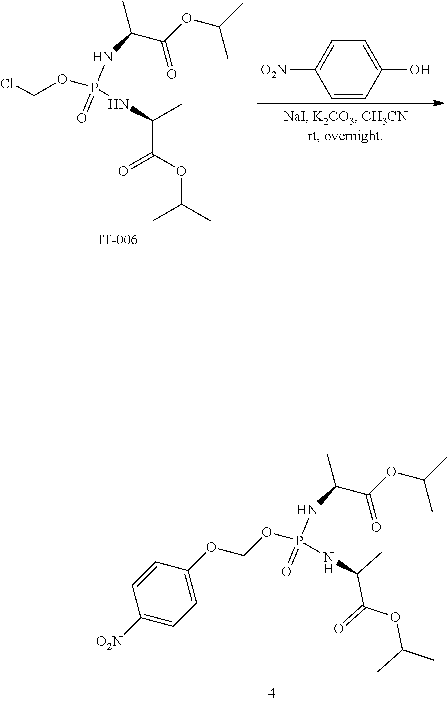 [SMILES: CC(C)OC(=O)[C@H](C)NP(=O)(N[C@@H](C)C(=O)OC(C)C)OCCl ; O=[N+]([O-])c1ccc(O)cc1 ; CC(C)OC(=O)[C@H](C)NP(=O)(N[C@@H](C)C(=O)OC(C)C)OCOc1ccc([N+](=O)[O-])cc1]

A mixture of IT-006 (see Example 2, 1.8 g, 4.83 mmol), 4-nitrophenol (1.01 g, 7.24 mmol), K₂CO₃ (1.34 g, 9.66 mmol) and NaI (145 mg, 0.97 mmol) in CH₃CN (27 mL) was stirred at room temperature overnight. The mixture was filtered and washed with CH₃CN. The solvent was removed in vacuo and the residue was purified by silica gel column chromatography and then preparative-TLC to give the title compound as a white solid.

### Example 5—Compound 5

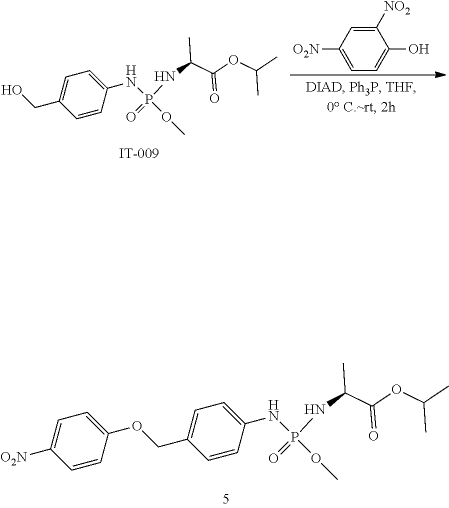

DIAD (3.91 g, 19.4 mmol) was added dropwise to a solution of IT-009 (see Example 3, 1.6 g, 4.84 mmol), 4-nitrophenol (1.01 g, 7.27 mmol) and Ph₃P (2.54 g, 9.68 mmol) in THF (30 mL) at 0° C. After addition, the mixture was stirred at room temperature for 2 hours. The solvent was removed in vacuo and the residue was purified by silica gel column chromatography, preparative-HPLC (CH₃CN/H₂O) and preparative-TLC to give the title compound as a slightly yellow solid.

### Example 6—Compound 6

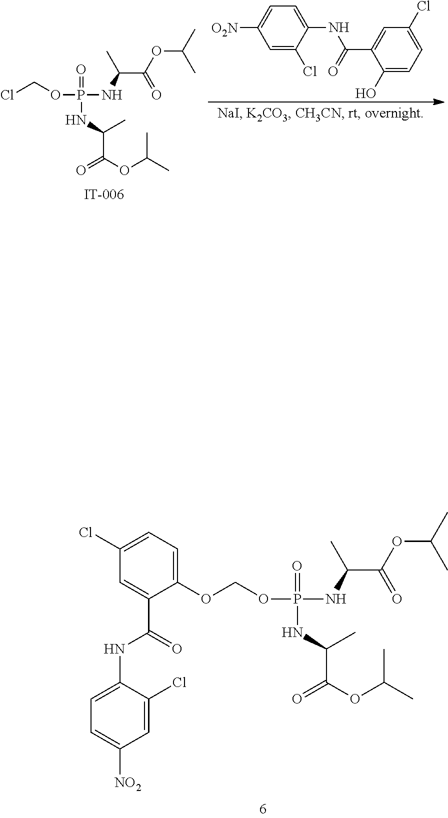

A mixture of IT-006 (see Example 2, 600 mg, 1.61 mmol), niclosamide (790 mg, 2.41 mmol), K₂CO₃ (445 mg, 3.22 mmol) and NaI (48 mg, 0.32 mmol) in CH₃CN (20 mL) was stirred at room temperature overnight. The mixture was filtered and washed with CH₃CN. The solvent was removed in vacuo and the residue was purified by preparative-HPLC (CH₃CN/H₂O) and then preparative-TLC to give the title compound as a white solid.

### Example 7—Compound 7

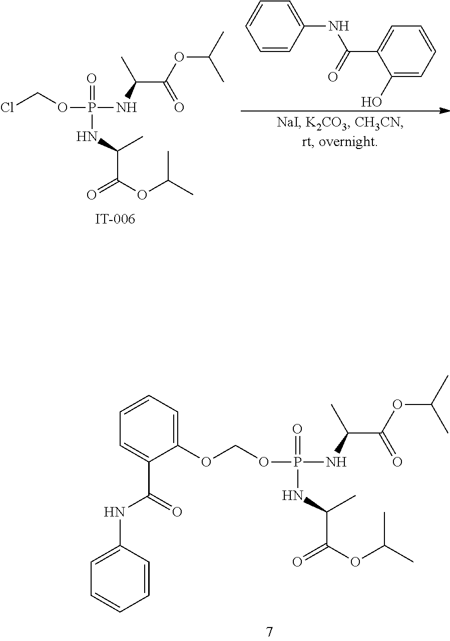

A mixture of IT-006 (see Example 2, 370 mg, 0.993 mmol), salicylanilide (317 mg, 1.49 mmol), K₂CO₃ (206 mg, 1.49 mmol) and NaI (30 mg, 0.2 mmol) in CH₃CN (7 mL) was stirred at room temperature overnight. The mixture was filtered and washed with CH₃CN. The solvent was removed and the residue was purified by silica gel column chromatography and then preparative-HPLC (CH₃CN/H₂O) to give the title compound as a yellow oil.

### Example 8—Compound 8

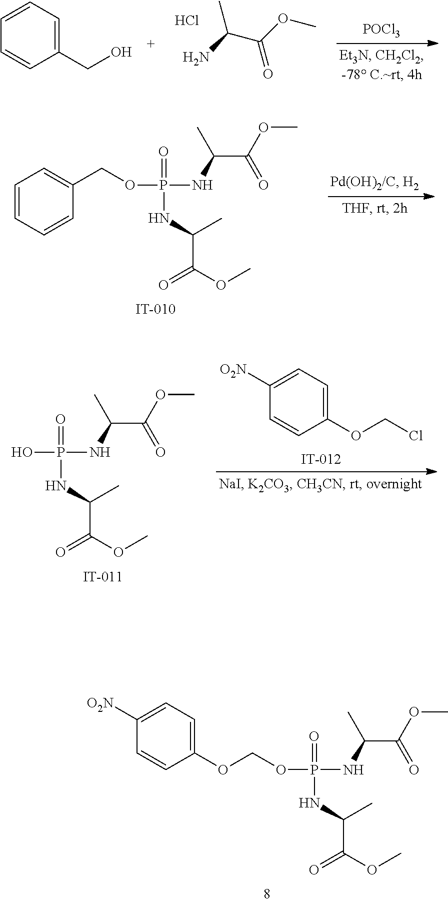

A mixture of benzyl alcohol (3.5 g, 32.4 mmol) and TEA (3.3 g, 32.4 mmol) was added dropwise to a solution of phosphoryl trichloride (5.0 g, 32.4 mmol) in DCM (150 mL) at −78° C. under Ar. The mixture was stirred at −78° C. for 30 min. L-alanine methyl ester hydrochloride (11.3 g, 80.9 mmol) was added and then TEA (16.4 g, 162 mmol) was added dropwise into the reaction mixture. After addition, the mixture was warmed to room temperature and stirred for 4 hours. The reaction mixture was quenched with water and extracted with DCM twice. The combined organic layers were dried (Na₂SO₄), filtered and the solvent was removed in vacuo. The residue was purified by silica gel column chromatography (EtOAc/petrol ether) to give IT-010 as colourless oil. A mixture of IT-010 (1.0 g, 2.8 mmol) and Pd(OH)₂/C (200 mg) in THF (30 mL) was stirred at room temperature under hydrogen atmosphere (balloon) for 2 hours. The reaction mixture was filtered and evaporated under reduced pressure to give IT-011 as colourless oil which was used for next step without purification. A mixture of IT-011 (112 mg, 0.42 mmol), IT-012 (see Example 20, 118 mg, 0.63 mmol), K₂CO₃ (116 mg, 0.84 mmol) and NaI (13 mg, 0.084 mmol) in CH₃CN (2 mL) was stirred at room temperature overnight.

The mixture was filtered and washed with CH₃CN. The solvent was removed in vacuo and the residue was purified by silica gel column chromatography to give the title compound as a white solid.

### Example 9—Compound 9

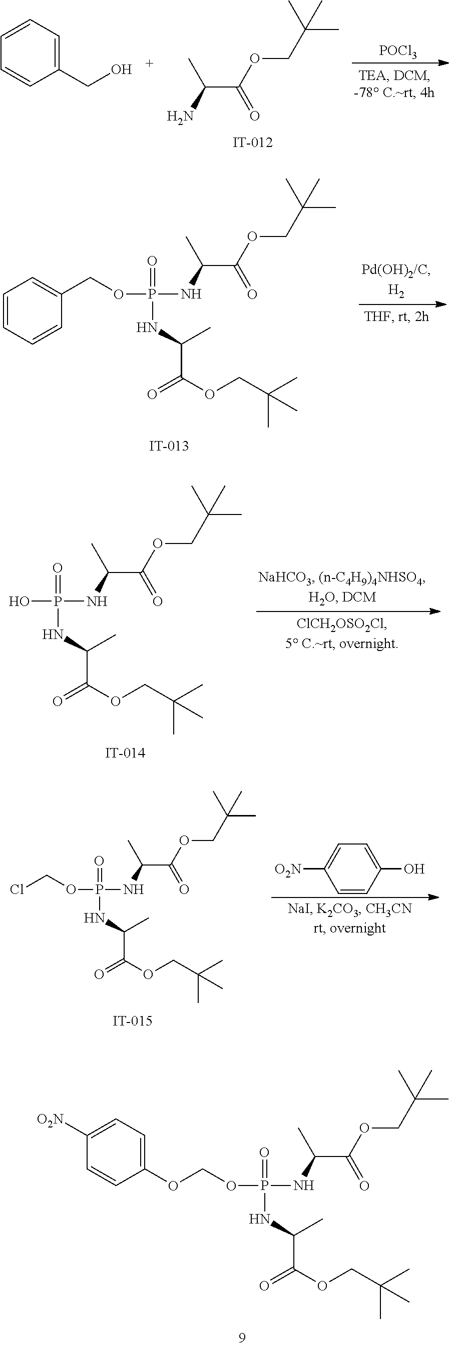

A mixture of benzyl alcohol (571 mg, 5.28 mmol) and TEA (594 mg, 5.87 mmol) was added dropwise to a solution of phosphoryl trichloride (900 mg, 5.87 mmol) in DCM (30 mL) at −78° C. under Ar. The mixture was stirred at −78° C. for 30 min. Then a solution of IT-012 (see Example 20, 2430 mg, 15.3 mmol) and TEA (2376 mg, 23.5 mmol) in DCM (3 mL) was added dropwise into the reaction mixture. After addition, the mixture was warmed to room temperature and stirred for 4 hours. The reaction mixture was quenched with water and extracted with DCM twice. The combined organic layers were dried (Na₂SO₄), filtered and then the solvent was removed in vacuo. The residue was purified by silica gel column chromatography (EtOAc/petrol ether) to give IT-013 as a colourless oil. A mixture of IT-013 (1.0 g, 2.13 mmol) and Pd(OH)₂/C (200 mg) in THF (30 mL) was stirred at room temperature under hydrogen atmosphere (balloon) for 2 hours. The reaction mixture was filtered and evaporated under reduced pressure to give IT-014 as a colourless oil which was used for next step without purification. Chloromethyl chlorosulfate (528 mg, 3.20 mmol) was added to a mixture of IT-014 (810 mg, 2.13 mmol), tetrabutylammonium hydrogen sulfate (72 mg, 0.21 mmol) and NaHCO₃ (716 mg, 8.53 mmol) in DCM (16 mL) and water (16 mL) at 5° C. After addition, the mixture was stirred at room temperature overnight. The mixture was diluted with DCM and washed with aqueous Na₂CO₃, water, 0.5 N HCl, water. The organic layer was dried over Na₂SO₄, filtered and then the solvent was removed in vacuo to give IT-015 as a colourless oil which was used in next step without purification. A mixture of IT-015 (200 mg, 0.47 mmol), 4-nitrophenol (97 mg, 0.70 mmol), K₂CO₃ (97 mg, 0.70 mmol) and NaI (14 mg, 0.09 mmol) in CH₃CN (3 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give the title compound as a colourless oil.

### Example 10—Compound 10

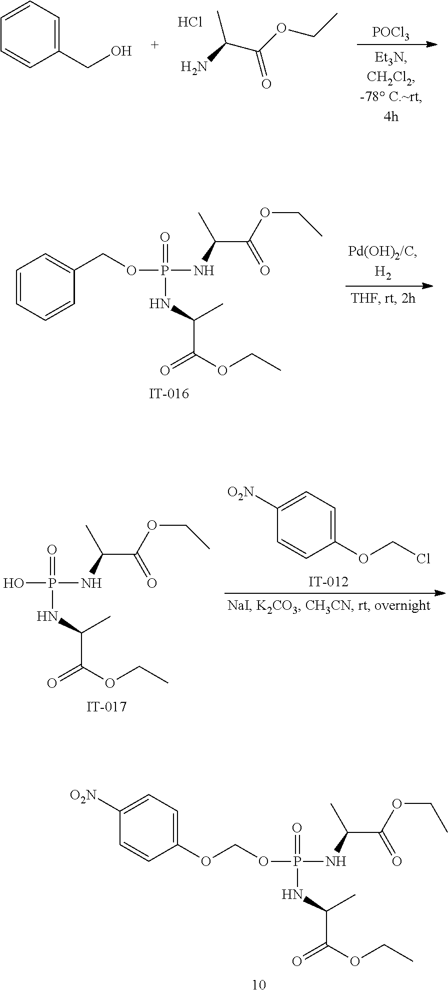

A mixture of benzyl alcohol (1.9 g, 17.6 mmol) and TEA (1.98 g, 19.6 mmol) was added dropwise to a solution of phosphoryl trichloride (3.0 g, 19.6 mmol) in DCM (90 mL) at −78° C. under Ar. The mixture was stirred at −78° C. for 30 min. L-alanine ethyl ester hydrochloride (7.51 g, 48.9 mmol) was added and then TEA (11.9 g, 117 mmol) was added dropwise into the reaction mixture. After addition, the mixture was warmed to room temperature and stirred for 4 hours. The reaction mixture was quenched with water and extracted with DCM twice. The combined organic layers were dried (Na₂SO₄), filtered and then the solvent was removed in vacuo. The residue was purified by silica gel column chromatography (EtOAc/petrol ether) to give IT-016 as a colourless oil. A mixture of IT-016 (200 mg, 0.518 mmol) and Pd(OH)₂/C (40 mg) in THF (8 ml) was stirred at room temperature under hydrogen atmosphere (balloon) for 2 hours. The reaction mixture was filtered and the solvent removed in vacuo to give IT-017 as a colourless oil which was used for next step without purification. A mixture of IT-017 (154 mg, 0.52 mmol), IT-012 (see Example 20, 146 mg, 0.78 mmol), K₂CO₃ (143 mg, 1.04 mmol) and NaI (15 mg, 0.10 mmol) in CH₃CN (2 mL) was stirred at room temperature overnight. The mixture was filtered and washed with CH₃CN. The solvent was removed in vacuo and the residue was purified by silica gel column chromatography to give the title compound as an off-white solid.

### Example 11—Compound 11

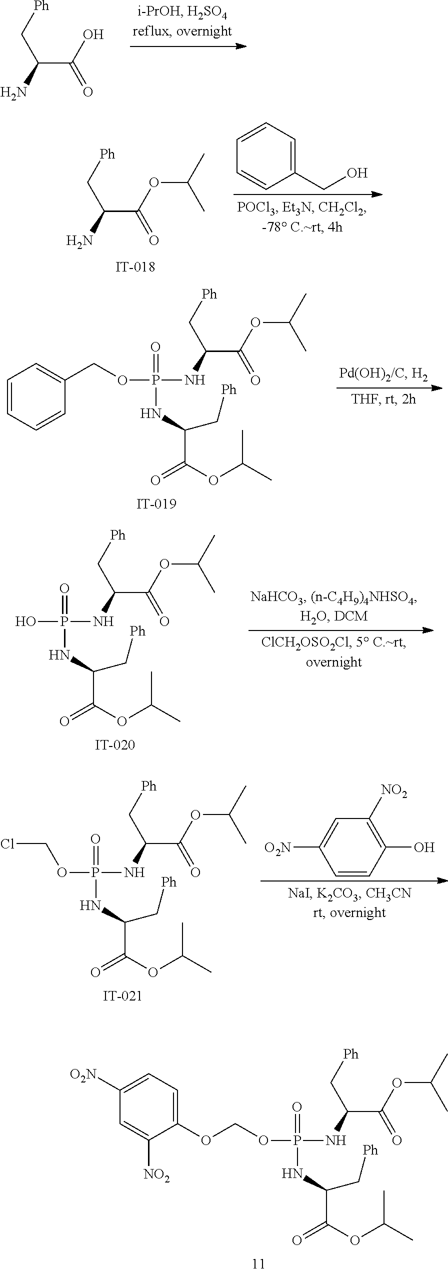

L-phenylalanine (10 g, 60.6 mmol) was dissolved in i-PrOH (100 mL) then concentrated H₂SO₄ (10 mL) was added slowly. The mixture was refluxed overnight before the solvent was removed in vacuo. To the residue, ice-water was added and then the solution was basified with aqueous NaOH. The resulting mixture was extracted with DCM twice.

The combined organic layers were dried (Na₂SO₄), filtered and then the solvent was removed in vacuo to give IT-018 as a colourless oil which could be used in next without purification. A mixture of benzyl alcohol (1.27 g, 11.7 mmol) and TEA (1.32 g, 13.0 mmol) was added dropwise to a solution of phosphoryl trichloride (2.0 g, 13.0 mmol) in DCM (60 mL) at −78° C. under Ar. The mixture was stirred at −78° C. for 30 min. Then a solution of IT-018 (6.76 g, 32.6 mmol) and TEA (5.28 g, 52.2 mmol) in DCM (5 mL) was added dropwise into the reaction mixture. After addition, the mixture was warmed to room temperature and stirred for 4 hours. The reaction mixture was quenched with water and extracted with DCM twice. The combined organic layers were dried (Na₂SO₄), filtered and then the solvent was removed in vacuo. The residue was purified by silica gel column chromatography (EtOAc/petrol ether) to give IT-019 as a colourless oil. A mixture of IT-019 (2.1 g, 3.71 mmol) and Pd(OH)₂/C (400 mg) in THF (60 mL) was stirred at room temperature under hydrogen atmosphere (balloon) for 2 hours. The reaction mixture was filtered and then the solvent was removed in vacuo to give IT-020 as a colourless oil which was used for next step without purification. Chloromethyl chlorosulfate (926 mg, 5.61 mmol) was added to a mixture of IT-020 (1.78 g, 3.74 mmol), tetrabutylammonium hydrogen sulfate (127 mg, 0.37 mmol) and NaHCO₃ (1.26 g, 15.0 mmol) in DCM (30 mL) and water (30 mL) at 5° C. After addition, the mixture was stirred at room temperature overnight. The mixture was diluted with DCM and washed with aqueous Na₂CO₃, water, 0.5 N HCl, water. The organic layer was dried over Na₂SO₄, filtered and then the solvent was removed in vacuo to give IT-021 as a colourless oil which was used in next step without purification. A mixture of IT-021 (600 mg, 1.15 mmol), 2,4-dinitrophenol (316 mg, 1.72 mmol), K₂CO₃ (237 mg, 1.72 mmol) and NaI (34 mg, 0.23 mmol) in CH₃CN (6 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give the title compound as yellow oil.

### Example 12—Compound 12

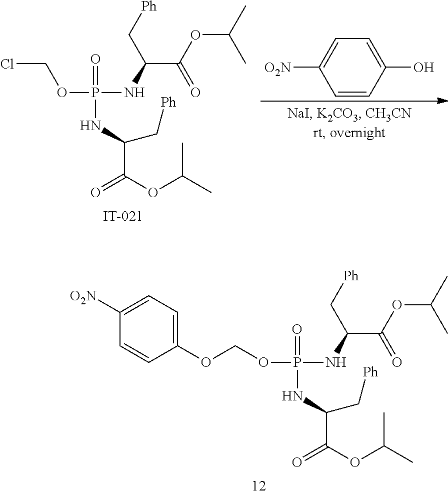

A mixture of IT-021 (see Example 11, 320 mg, 0.61 mmol), 4-nitrophenol (127 mg, 0.92 mmol), K₂CO₃ (126 mg, 0.92 mmol) and NaI (18 mg, 0.12 mmol) in CH₃CN (6 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give the title compound as a colourless oil.

### Example 13—Compound 13

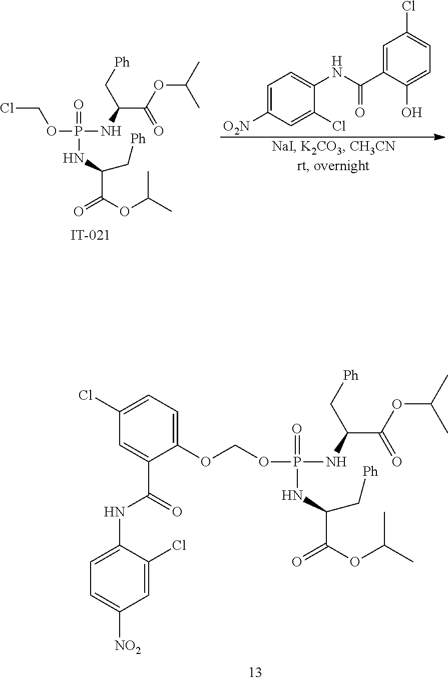 [SMILES: CC(C)OC(=O)[C@H](Cc1ccccc1)NP(=O)(N[C@@H](Cc1ccccc1)C(=O)OC(C)C)OCCl ; O=C(Nc1ccc([N+](=O)[O-])cc1Cl)c1cc(Cl)ccc1O ; CC(C)OC(=O)[C@H](Cc1ccccc1)NP(=O)(N[C@@H](Cc1ccccc1)C(=O)OC(C)C)OCOc1ccc(Cl)cc1C(=O)Nc1ccc([N+](=O)[O-])cc1Cl]

A mixture of IT-021 (see Example 11, 320 mg, 0.61 mmol), niclosamide (301 mg, 0.92 mmol), K₂CO₃ (126 mg, 0.92 mmol) and NaI (18 mg, 0.12 mmol) in CH₃CN (6 mL) was stirred at room temperature overnight. The mixture was filtered and then the solvent was removed in vacuo. The residue was purified by preparative-HPLC (CH₃CN/H₂O) and the crude product was rinsed with EtOH to give pure title compound as an off-white solid.

### Example 14—Compound 14

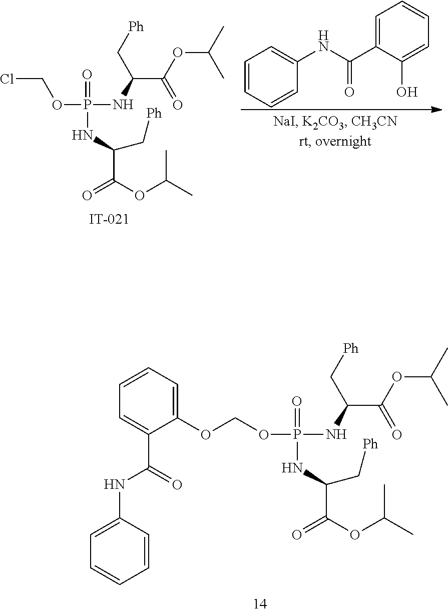

A mixture of IT-021 (see Example 11, 320 mg, 0.61 mmol), salicylanilide (196 mg, 0.92 mmol), K₂CO₃ (126 mg, 0.92 mmol) and NaI (18 mg, 0.12 mmol) in CH₃CN (6 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give the title compound as a yellow oil.

### Example 15—Compound 15

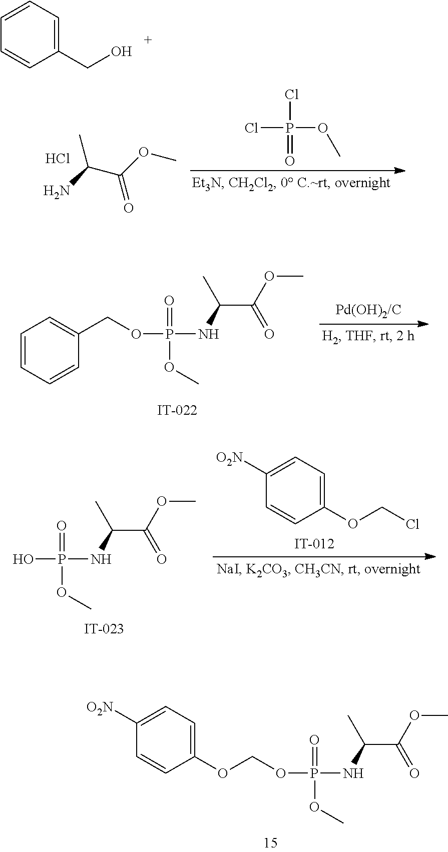

A mixture of benzyl alcohol (1.31 g, 12.1 mmol) and TEA (1.36 g, 13.4 mmol) was added dropwise to a solution of methyl phosphorodichloridate (2.0 g, 13.4 mmol) in DCM (40 mL) at 0° C. under nitrogen. After addition, the reaction was stirred at room temperature for 30 min before it was re-cooled to 0° C. L-alanine methyl ester hydrochloride (2.25 g, 16.1 mmol) was added to the reaction and then TEA (4.08 g, 40.3 mmol) was added dropwise. The reaction mixture was stirred at room temperature overnight before being quenched with water. The resulting mixture was extracted with DCM twice. The combined organic layers were dried over Na₂SO₄, filtered and then the solvent was removed in vacuo. The residue was purified by silica gel column chromatography to give IT-022 as a colourless oil. A mixture of IT-022 (200 mg, 0.7 mmol) and Pd(OH)₂/C (40 mg) in THF (6 mL) was stirred at room temperature under hydrogen atmosphere (balloon) for 2 hours. The reaction mixture was filtered and the solvent was removed from the in vacuo to give IT-023 as a colourless oil. A mixture of IT-023 (69 mg, 0.35 mmol), IT-012 (see Example 20, 98 mg, 0.52 mmol), K₂CO₃ (96 mg, 0.7 mmol) and NaI (10.5 mg, 0.07 mmol) in CH₃CN (2 mL) was stirred at room temperature overnight. The mixture was filtered and washed with CH₃CN. The solvent was removed from the filtrate in vacuo and the residue was purified by silica gel column chromatography to give the title compound as a colourless oil.

### Example 16—Compound 16

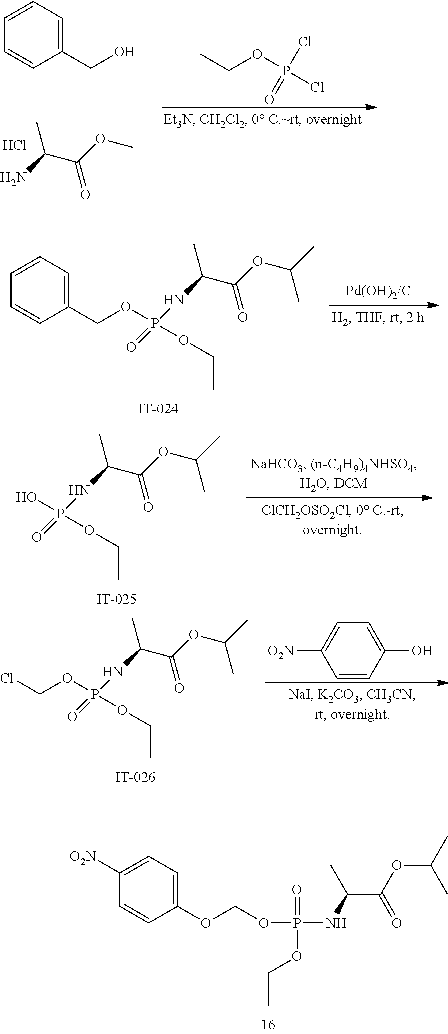

A mixture of benzyl alcohol (1.6 g, 14.7 mmol) and TEA (2.24 g, 22.1 mmol) was added dropwise to a solution of ethyl phosphorodichloridate (3.0 g, 18.4 mmol) in DCM (50 mL) at 0° C. under nitrogen. After addition, the reaction was stirred at room temperature for 30 min before re-cooled to 0° C. L-Alanine isopropyl ester hydrochloride (3.7 g, 22.1 mmol) was added to the reaction and then TEA (5.59 g, 55.2 mmol) was added dropwise. The reaction mixture was stirred at room temperature overnight before it was quenched with water. The resulting mixture was extracted with DCM twice. The combined organic layers were dried over Na₂SO₄, filtered and then the solvent was removed in vacuo. The residue was purified by silica gel column chromatography to give IT-024 as a colourless oil. A mixture of IT-024 (500 mg, 1.52 mmol) and Pd(OH)₂/C (100 mg) in THF (20 mL) was stirred at room temperature under hydrogen atmosphere (balloon) for 2 hours. The reaction mixture was filtered and then the solvent was removed from the filtrate in vacuo to give IT-025 as a colourless oil. Chloromethyl chlorosulfate (376 mg, 2.28 mmol) was added to a mixture of IT-025 (363 mg, 1.52 mmol), tetrabutylammonium hydrogen sulfate (52 mg, 0.152 mmol) and NaHCO₃ (510 mg, 6.07 mmol) in DCM (10 mL) and water (10 mL) at 0° C. After addition, the mixture was stirred at room temperature overnight. The mixture was extracted with DCM twice. The combined organic layers were dried over Na₂SO₄, filtered and then the solvent was removed in vacuo to give IT-026 as a colourless oil which was used in next step without purification. A mixture of IT-026, 4-nitrophenol (211 mg, 1.52 mmol), K₂CO₃ (315 mg, 2.28 mmol) and NaI (46 mg, 0.3 mmol) in CH₃CN (5 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give the title compound as a slightly yellow oil.

### Example 17—Compound 17

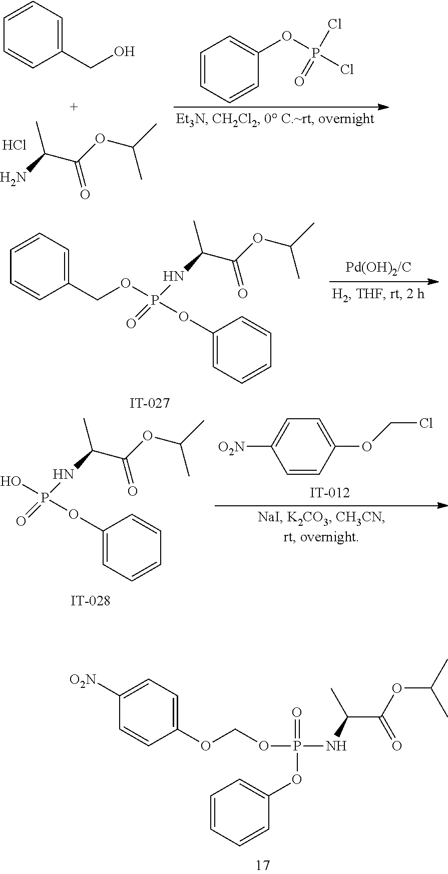

A mixture of benzyl alcohol (1.23 g, 11.4 mmol) and TEA (1.73 g, 17.1 mmol) was added dropwise to a solution of phenyl phosphorodichloridate (3.0 g, 14.2 mmol) in DCM (50 mL) at 0° C. under nitrogen. After addition, the reaction was stirred at room temperature for 30 min before it was re-cooled to 0° C. L-Alanine isopropyl ester hydrochloride (2.9 g, 17.1 mmol) was added to the reaction and then TEA (4.32 g, 42.7 mmol) was added dropwise. The reaction mixture was stirred at room temperature overnight before being quenched with water. The resulting mixture was extracted with DCM twice. The combined organic layers were dried over Na₂SO₄, filtered and then the solvent was removed in vacuo. The residue was purified by silica gel column chromatography to give IT-027 as a colourless oil. A mixture of IT-027 (1.0 g, 2.65 mmol) and Pd(OH)₂/C (200 mg) in THF (20 mL) was stirred at room temperature under hydrogen atmosphere (balloon) for 2 hours. The reaction mixture was filtered and then the solvent was removed from the filtrate in vacuo to give IT-028 as a colourless oil. A mixture of IT-028 (381 mg, 1.33 mmol), IT-012 (see Example 20, 1245 mg, 6.64 mmol), K₂CO₃ (368 mg, 2.66 mmol) and NaI (41 mg, 0.27 mmol) in CH₃CN (8 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give the title compound as a colourless oil.

### Example 18—Compound 18

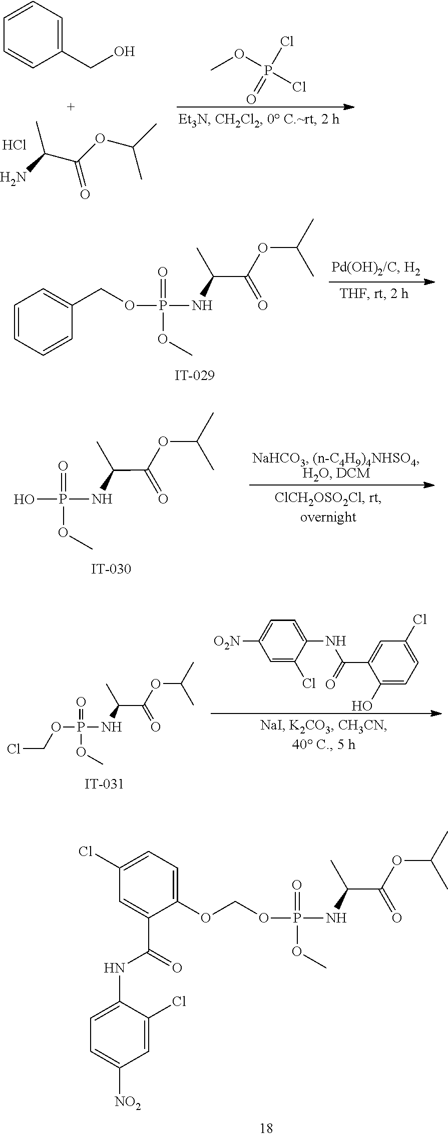

A mixture of benzyl alcohol (2.18 g, 20.1 mmol) and TEA (2.04 g, 20.1 mmol) was added dropwise to a solution of methyl phosphorodichloridate (3.0 g, 20.1 mmol) in DCM (60 ml) at 0° C. under nitrogen. After addition, the reaction was stirred at room temperature for 30 min before re-cooled to 0° C. L-Alanine isopropyl ester hydrochloride (3.71 g, 22.2 mmol) was added to the reaction and then TEA (6.12 g, 60.4 mmol) was added dropwise. The reaction mixture was stirred at room temperature for 2 hours before it was quenched with water. The resulting mixture was extracted with DCM twice, then the combined organic layers were dried over Na₂SO₄, filtered and the solvent was removed in vacuo. The residue was purified by silica gel column chromatography to give IT-029 as a colourless oil. A mixture of IT-029 (1.0 g, 3.17 mmol) and Pd(OH)₂/C (100 mg) in THF (30 mL) was stirred at room temperature under hydrogen atmosphere (balloon) for 2 hours. The reaction mixture was filtered and then the solvent was removed in vacuo to give IT-030 as a colourless oil. Chloromethyl chlorosulfate was added (3146 mg, 19.1 mmol) to a mixture of IT-030 (715 mg, 3.2 mmol), tetrabutylammonium hydrogen sulfate (109 mg, 0.32 mmol) and NaHCO₃ (3023 mg, 38.1 mmol) in DCM (20 mL) and water (20 mL) at room temperature. The mixture was stirred at room temperature overnight then extracted with DCM 3 times. The combined organic layers were dried over Na₂SO₄, filtered and then the solvent was removed in vacuo to give IT-031 as a slightly yellow oil which was used in next step without purification. A mixture of IT-031 (240 mg, 0.88 mmol), niclosamide (430 mg, 1.31 mmol), K₂CO₃ (363 mg, 2.63 mmol) and NaI (66 mg, 0.44 mmol) in CH₃CN (15 mL) was stirred at 40° C. for 5 hours. The mixture was cooled and filtered. The solvent was removed from the filtrate in vacuo and the residue was purified by silica gel column chromatography to give the title compound as a grey solid.

### Example 19—Compound 19

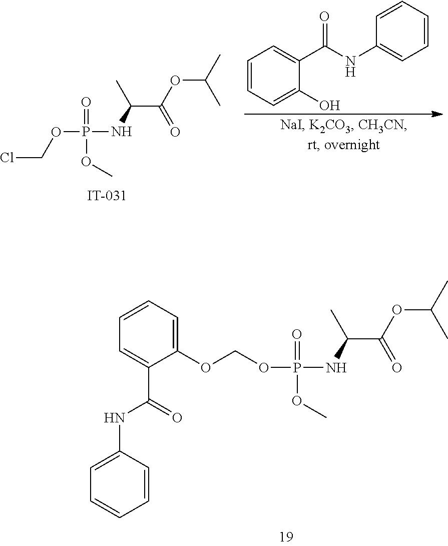

A mixture of IT-031 (see Example 18, 300 mg, 1.09 mmol), salicylanilide (350 mg, 1.64 mmol), K₂CO₃ (453 mg, 3.28 mmol) and NaI (82 mg, 0.55 mmol) in CH₃CN (9 mL) was stirred at room temperature overnight. The mixture was filtered and washed with EtOAc. The solvent was removed from the filtrate in vacuo and the residue was purified by silica gel column chromatography to give the title compound as a slightly yellow solid.

### Example 20—Compound 20

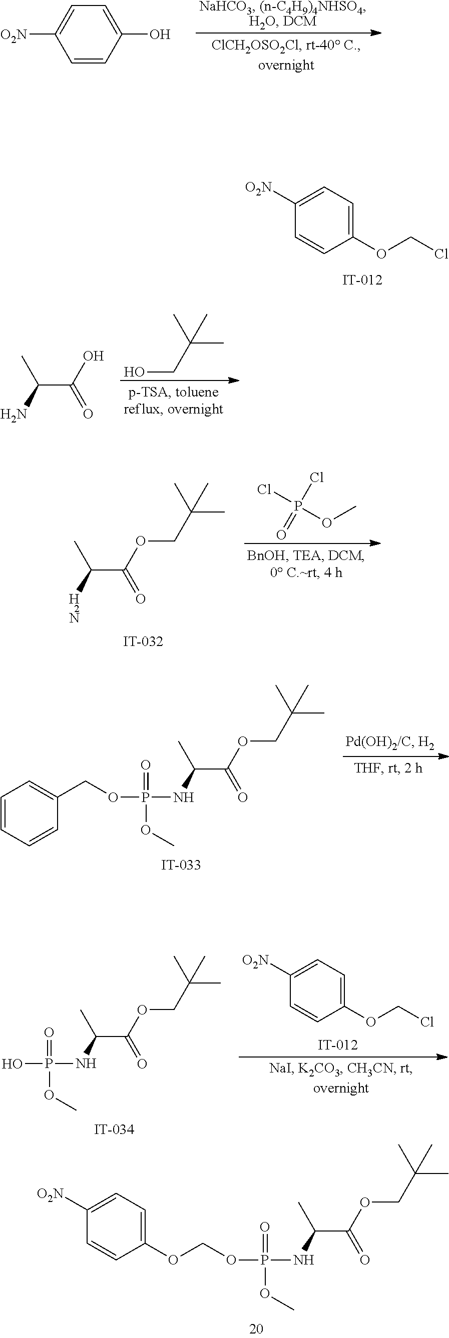

Chloromethyl chlorosulfate (10.7 g, 64.7 mmol) was added to a mixture of 4-nitrophenol (3.0 g, 21.6 mmol), tetrabutylammonium hydrogen sulfate (732 mg, 2.16 mmol) and NaHCO₃ (18.1 g, 216 mmol) in DCM (90 mL) and water (90 mL) at room temperature. The mixture was stirred at 40° C. overnight. The mixture was cooled, diluted with water and extracted with DCM twice. The combined organic layers were dried over Na₂SO₄, filtered and the solvent was removed in vacuo. The residue was purified by silica gel column chromatography to give IT-012 as a colourless oil. A mixture of L-alanine (60.0 g, 0.673 mol), 2,2-dimethylpropan-1-ol (59.4 g, 0.673 mol) and p-toluenesulfonic acid (p-TSA) monohydrate (140.9 g, 0.741 mol) in toluene (1000 mL) was heated to reflux overnight, using a Dean-Stark apparatus. The reaction mixture was cooled and the precipitate was collected by filtration to give the product (80 g) as the p-toluenesulfonate salt. The p-toluenesulfonate salt (40 g) was dissolved in water and basified to pH=9-10 by aqueous Na₂CO₃. The resulting solution was extracted with DCM 3 times. The combined organic layers were washed with brine, dried over Na₂SO₄ and the solvent was removed in vacuo to give free IT-032 as a colourless oil. A mixture of benzyl alcohol (1.5 g, 13.9 mmol) and TEA (1.4 g, 13.9 mmol) was added dropwise to a solution of methyl phosphorodichloridate (2.1 g, 13.9 mmol) in DCM (30 ml) at 0° C. under nitrogen. After addition, the reaction was stirred at room temperature for 30 min before it was re-cooled to 0° C. A solution of IT-032 (2.4 g, 15.3 mmol) and TEA (2.1 g, 20.8 mmol) in DCM (10 mL) was added dropwise to the reaction. The reaction mixture was stirred at room temperature for 4 hours then quenched with water. The resulting mixture was extracted with DCM twice. The combined organic layers were dried over Na₂SO₄, filtered and the solvent was removed in vacuo. The residue was purified by silica gel column chromatography to give IT-033 as a colourless oil. A mixture of IT-033 (100 mg, 0.29 mmol) and Pd(OH)₂/C (20 mg) in THF (5 mL) was stirred at room temperature under hydrogen atmosphere (balloon) for 2 hours. The reaction mixture was filtered and then the solvent from the filtrate was removed in vacuo to give IT-034 as a colourless oil. A mixture of IT-034 (74 mg, 0.29 mmol), IT-012 (82 mg, 0.44 mmol), K₂CO₃ (81 mg, 0.58 mmol) and NaI (13 mg, 0.088 mmol) in CH₃CN (2 mL) was stirred at room temperature overnight. The mixture was filtered and washed with CH₃CN. The solvent from the filtrate was removed in vacuo and the residue was purified by silica gel column chromatography to give the title compound as a slightly yellow oil.

### Example 21—Compound 21

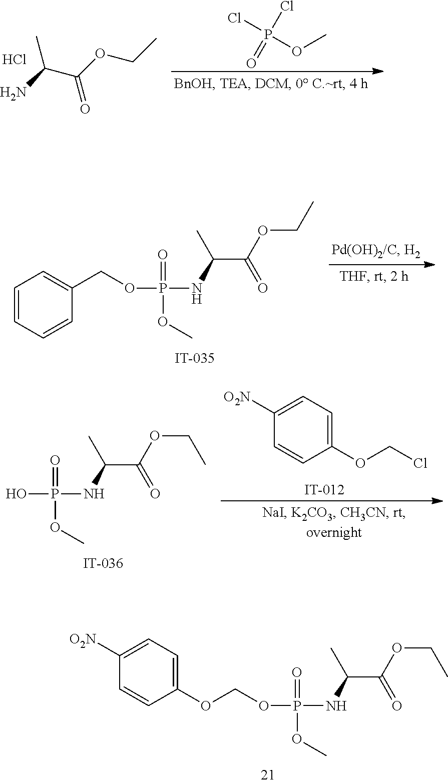

A mixture of benzyl alcohol (1.5 g, 13.9 mmol) and TEA (1.4 g, 13.9 mmol) was added dropwise to a solution of methyl phosphorodichloridate (2.1 g, 13.9 mmol) in DCM (30 ml) at 0° C. under nitrogen. After addition, the reaction was stirred at room temperature for 30 min then re-cooled to 0° C. Ethyl L-alaninate hydrochloride (2.35 g, 15.3 mmol) was added to the reaction and then TEA (4.2 g, 41.7 mmol) was added dropwise. The reaction mixture was stirred at room temperature for 4 hours then quenched with water.

The resulting mixture was extracted with DCM twice. The combined organic layers were dried over Na₂SO₄, filtered and the solvent was removed in vacuo. The residue was purified by silica gel column chromatography to give IT-035 as a colourless oil. A mixture of IT-035 (200 mg, 0.66 mmol) and Pd(OH)₂/C (40 mg) in THF (8 mL) was stirred at room temperature under hydrogen atmosphere (balloon) for 2 hours. The reaction mixture was filtered and then the solvent from the filtrate was removed in vacuo to give IT-036 as a colourless oil. A mixture of IT-036 (141 mg, 0.67 mmol), IT-012 (see Example 20, 188 mg, 1.0 mmol), K₂CO₃ (185 mg, 1.3 mmol) and NaI (30 mg, 0.2 mmol) in CH₃CN (3 mL) was stirred at room temperature overnight. The mixture was filtered and washed with CH₃CN. The solvent from the filtrate was removed in vacuo and the residue was purified by silica gel column chromatography to give the title compound as a slightly yellow oil.

### Example 22—Compound 22

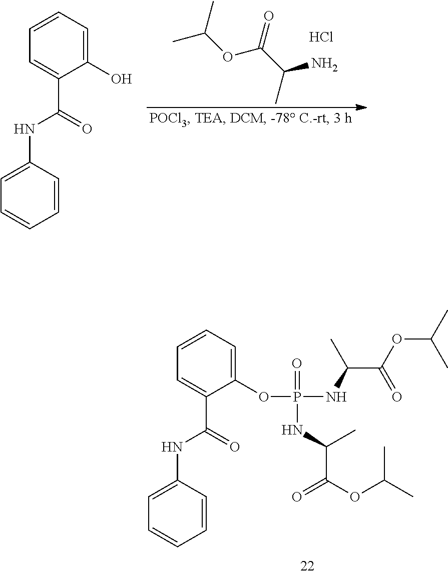

TEA (1.9 g, 18.8 mmol) was added dropwise to a solution of POCl₃ (2.85 g, 18.8 mmol) and salicylanilide (4.0 g, 18.8 mmol) in dry DCM (100 ml) at −78° C. under an atmosphere of Argon. The mixture was stirred at −78° C. for 30 min. L-Alanine isopropyl ester hydrochloride (7.9 g, 46.9 mmol) was added to the reaction and then TEA (11.4 g, 112.7 mmol) was added dropwise at −78° C. The reaction mixture was stirred at room temperature for 3 hours before it was quenched with water. The resulting mixture was extracted with DCM twice. The combined organic layers were dried over Na₂SO₄, filtered and then the solvent was removed in vacuo. The residue was purified by silica gel column chromatography twice (EtOAc/petroleum ether=1/3 to 1/2) to give the title compound as a slightly yellow oil.

### Example 23—Compound 23

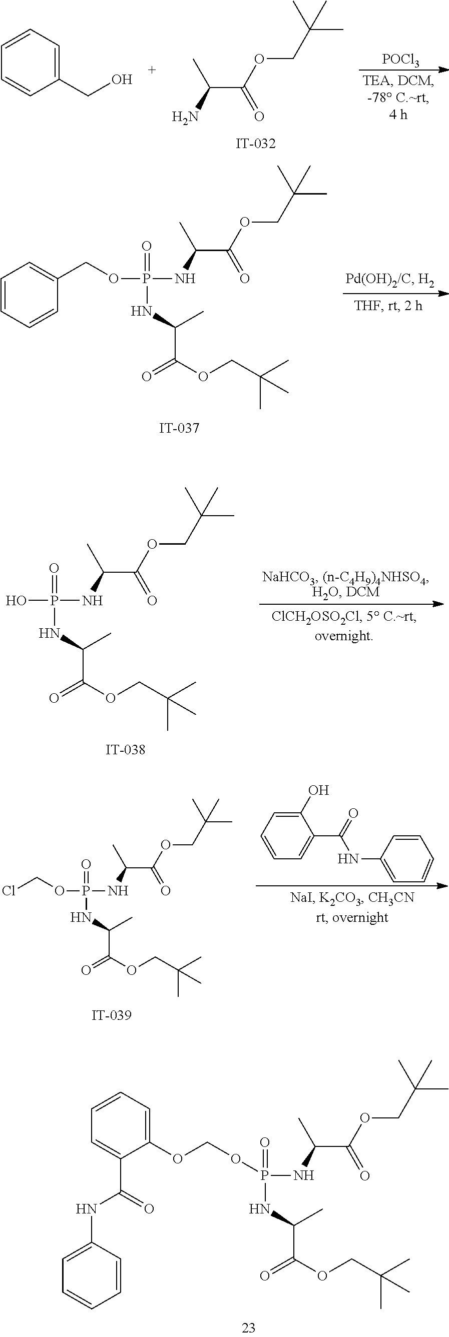

A mixture of phenylmethanol (571 mg, 5.28 mmol) and TEA (594 mg, 5.87 mmol) was added dropwise to a solution of phosphoryl trichloride (900 mg, 5.87 mmol) in DCM (30 mL) at −78° C. under inert conditions. The mixture was stirred at the same temperature for 30 minutes then a solution of IT-032 (see Example 20, 2430 mg, 15.3 mmol) and TEA (2376 mg, 23.5 mmol) in DCM (3 mL) was added dropwise. The mixture was then warmed to room temperature and stirred for another 4 hours. The reaction mixture was quenched with water and extracted with DCM twice. The combined organic layers were dried over Na₂SO₄, filtered and then the solvent was removed in vacuo. The residue was purified by silica gel column chromatography (EtOAc/petrol ether) to give IT-037 as colorless oil. A mixture of IT-037 (1.0 g, 2.13 mmol) and Pd(OH)₂/C (200 mg) in THF (30 mL) was stirred at room temperature under hydrogen atmosphere (balloon) for 2 hours. The reaction mixture was filtered and the solvent evaporated under reduced pressure to give IT-038 as colorless oil which was used for next step without purification. Chloromethyl chlorosulfate (528 mg, 3.20 mmol) was added to a mixture of IT-038 (810 mg, 2.13 mmol), tetrabutylammonium hydrogen sulfate (72 mg, 0.21 mmol) and NaHCO₃ (716 mg, 8.53 mmol) in DCM (16 mL) and water (16 mL) at 5° C. After addition, the mixture was stirred at room temperature overnight. The mixture was then diluted with DCM and successively washed with saturated aqueous Na₂CO₃ solution, water, then 0.5 N HCl and water. The organic layer was dried over Na₂SO₄, filtered and then the solvent was removed in vacuo to give IT-039 as colorless oil which was used in next step without purification. A mixture of IT-039 (400 mg, 0.93 mmol), 2-hydroxy-N-phenylbenzamide (298 mg, 1.40 mmol), K₂CO₃ (193 mg, 1.40 mmol) and NaI (28 mg, 0.18 mmol) in CH₃CN (10 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography to give compound 23 as colorless oil.

### Example 24—Compound 24

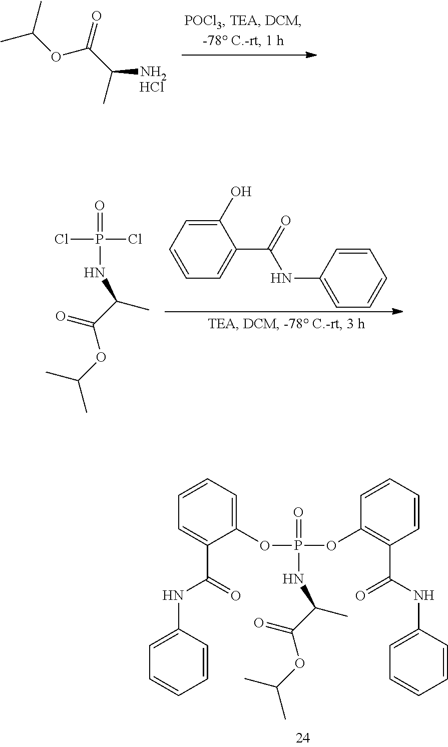

TEA (1.2 g, 12 mmol) was added dropwise to a solution of POCl₃ (912 mg, 6 mmol) and L-Alanine isopropyl ester hydrochloride (1 g, 6 mmol) in dry DCM (30 ml) at −78° C. under inert conditions. The mixture was stirred at −78° C. for 1 hour. Salicylanilide (2.6 g, 12 mmol) was then added followed by the addition of TEA (1.2 g, 12 mmol) dropwise at −78° C. The resulting mixture was stirred at room temperature for another 3 hours before it was quenched with water. The solvent was removed in vacuo and the residue was purified by prep-HPLC to give compound 24 as white solid.

### Example 25—Compound 25

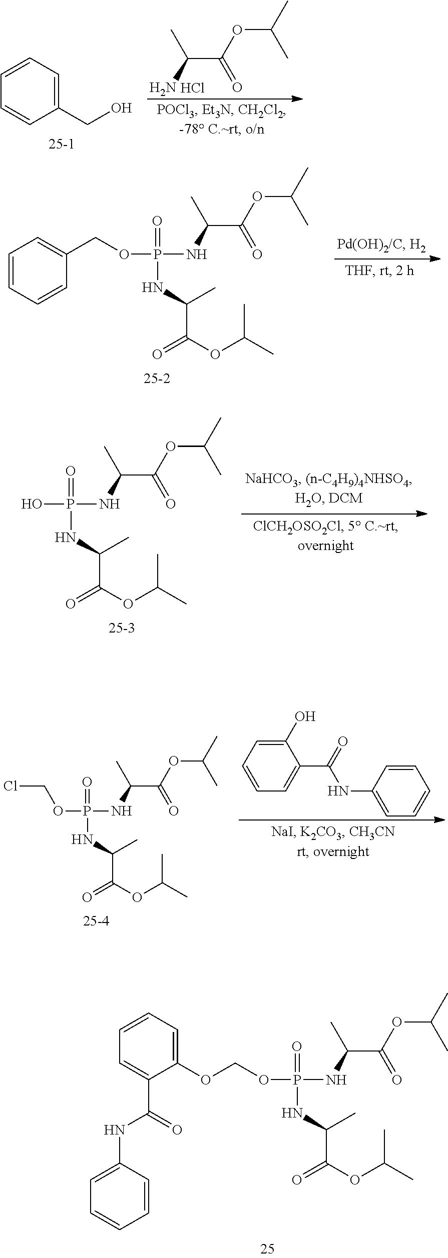

A mixture of phenylmethanol (633 mg, 5.86 mmol) and TEA (658 mg, 6.51 mmol) was added dropwise to a solution of phosphoryl trichloride (1.0 g, 6.51 mmol) in DCM (30 mL) at −78° C. under inert conditions. The mixture was stirred at −78° C. for 30 minutes. Then a solution of (S)-isopropyl 2-aminopropanoate hydrochloride (2.73 g, 16.28 mmol) and TEA (3.4 g, 33.85 mmol) were added dropwise into the reaction mixture separately. After addition, the mixture was warmed to room temperature and stirred for 4 hours. The reaction mixture was quenched with water and extracted with DCM twice.

The combined organic layers were dried over Na₂SO₄, filtered and then the solvent was removed in vacuo. The residue was purified by silica gel column chromatography (EA/PE=1/2) to give 25-2 as colorless oil. A mixture of 25-2 (1.85 g, 4.47 mmol) and Pd(OH)₂/C (600 mg) in THF (30 mL) was stirred at room temperature under hydrogen atmosphere (balloon) for 2 hours. The reaction mixture was filtered and the solvent evaporated under reduced pressure to give 25-3 as colorless oil which was used for next step without purification. Chloromethyl chlorosulfate (1.16 g, 7.05 mmol) was added to a mixture of 25-3 (1.53 g, 4.7 mmol), tetrabutylammonium hydrogen sulfate (160 mg, 0.47 mmol) and NaHCO₃ (1.6 g, 18.8 mmol) in DCM (20 mL) and water (20 mL) at 5° C. After addition, the mixture was stirred at room temperature overnight. The mixture was diluted with DCM and washed with aqueous Na₂CO₃, water, 0.5 N HCl, water. The organic layer was dried over Na₂SO₄, filtered and concentrated under reduced pressure to give 25-4 as colorless oil which was used in next step without purification. A mixture of 25-4 (900 mg, 2.42 mmol), 2-hydroxy-N-phenylbenzamide (773 mg, 3.63 mmol), K₂CO₃ (501 mg, 1.5 mmol) and NaI (72 mg, 0.48 mmol) in CH₃CN (10 mL) was stirred at room temperature overnight. The mixture was diluted with water and extracted with EtOAc twice. The combined organic layers were dried over Na₂SO₄, filtered and concentrated. The residue was purified by silica gel column chromatography (EA/PE=1/2) to give 25 as colorless oil.

### Example 26—Compound 26

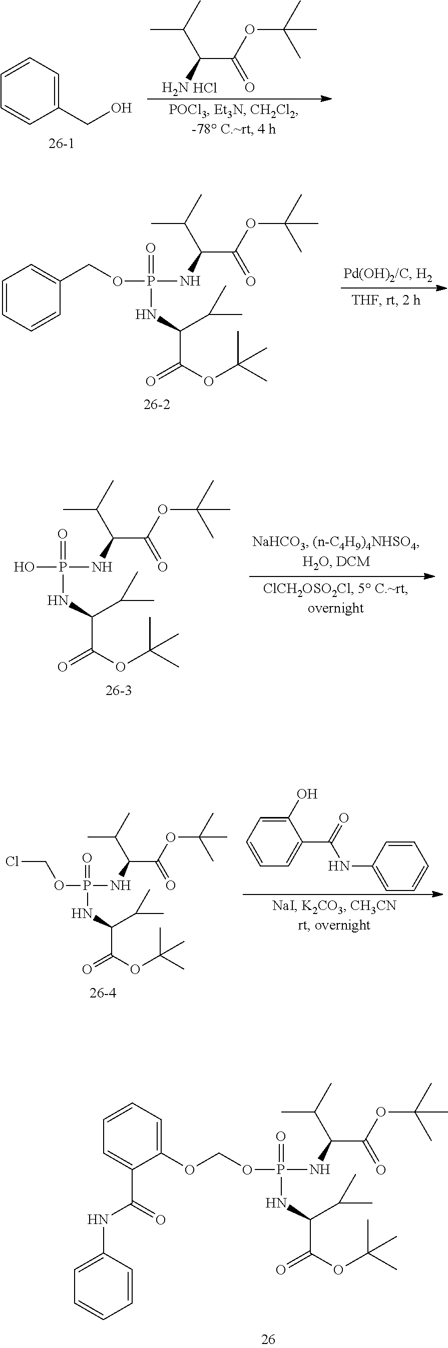

A mixture of phenylmethanol (633 mg, 5.86 mmol) and TEA (658 mg, 6.51 mmol) was added dropwise to a solution of phosphoryl trichloride (1.0 g, 6.51 mmol) in DCM (30 mL) at −78° C. under inert conditions. The mixture was stirred at −78° C. for 30 minutes. Then a solution of (S)-tert-butyl 2-amino-3-methylbutanoate hydrochloride (3.0 g, 14.3 mmol) and TEA (3.02 g, 29.9 mmol) were separately added dropwise into the reaction mixture. After addition, the mixture was warmed to room temperature and stirred for 4 hours. The reaction mixture was quenched with water and extracted with DCM twice. The combined organic layers were dried (Na₂SO₄), filtered and then the solvent was removed in vacuo. The residue was purified by silica gel column chromatography (EA/PE=1/2) to give 26-2 as colorless oil. A mixture of 26-2 (2.1 g, 4.2 mmol) and Pd(OH)₂/C (500 mg) in THF (30 mL) was stirred at room temperature under hydrogen atmosphere (balloon) for 2 hours. The reaction mixture was filtered and evaporated under reduced pressure to give 26-3 (as colorless oil which was used for next step without purification. Chloromethyl chlorosulfate was added (1.49 mg, 9.03 mmol) to a mixture of 26-3 (2.46 g, 6.02 mmol), tetrabutylammonium hydrogen sulfate (204 mg, 0.6 mmol) and NaHCO₃ (2.02 g, 24.08 mmol) in DCM (30 mL) and water (30 mL) at 5° C. After addition, the mixture was stirred at room temperature overnight. The mixture was diluted with DCM and washed with aqueous Na₂CO₃, water, 0.5 N HCl, water. The organic layer was dried over Na₂SO₄, filtered and concentrated under reduced pressure to give 26-4 as colorless oil which was used in next step without purification. A mixture of 26-4 (2.1 g, 4.73 mmol), 2-hydroxy-N-phenylbenzamide (1.51 g, 7.1 mmol), K₂CO₃ (980 mg, 7.1 mmol) and NaI (142 mg, 0.95 mmol) in CH₃CN (20 mL) was stirred at room temperature overnight. The mixture was diluted with water and extracted with EtOAc twice. The combined organic layers were dried over Na₂SO₄, filtered and concentrated. The residue was purified by silica gel column chromatography (EA/PE=1/2) to give 26 as colorless oil.

### Example 27—Compound 27

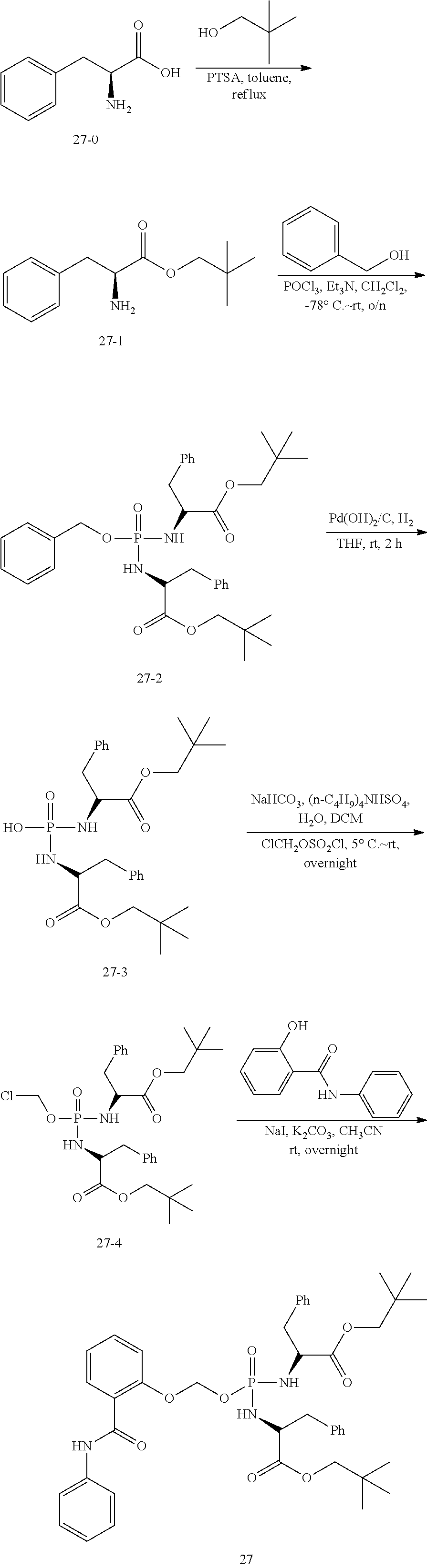

A mixture of 27-0 (3.3 g, 20 mmol), 2,2-dimethylpropan-1-ol (3.5 g, 40 mmol), and p-toluenesulfonic acid (PTSA) monohydrate (4.1 g, 24 mmol) in toluene (50 mL) was heated with a Dean-Stark trap, and kept at reflux temperature overnight. The reaction mixture was cooled and then the solvent was removed in vacuo. The residue was dissolved in DCM and basified to pH 9-10 by saturated aqueous Na₂CO₃. The resulting solution was extracted with DCM 3 times. The combined organic layers were washed with brine, dried over Na₂SO₄ and then the solvent was removed in vacuo. The residue was purified by silica gel column chromatography (EtOAc/petrol ether=1/10 to 1/1) to give 27-1 as yellow oil. A mixture of phenylmethanol (380 mg, 3.52 mmol) and TEA (395 mg, 3.91 mmol) was added dropwise to a solution of phosphoryl trichloride (600 mg, 3.91 mmol) in DCM (15 mL) at −78° C. under Ar and stirred for 30 min. Separately, a solution of 27-1 (2.2 g, 9.38 mmol) in DCM (5 mL) and then TEA (1.42 g, 14.01 mmol) in DCM (5 mL) were added dropwise into the reaction mixture at −78° C. After addition, the mixture was warmed to room temperature and stirred for 4 hours. The reaction mixture was quenched with water and extracted with DCM twice. The combined organic layers were dried (Na₂SO₄), filtered and then the solvent was removed in vacuo. The residue was purified by silica gel column chromatography (EtOAc/petrol ether=1/10 to 1/1) to give 27-2 as a colourless oil. A mixture of 27-2 (1.3 g, 2.09 mmol) and Pd(OH)₂/C (300 mg) in THF (20 mL) was stirred at room temperature under hydrogen atmosphere (balloon) for 2 hours. The reaction mixture was filtered and the filtrate then the solvent was removed in vacuo to give 27-3 as a colourless oil. Chloromethyl chlorosulfate (436 mg, 2.64 mmol) was added to a mixture of 27-3 (938 mg, 1.76 mmol), tetrabutylammonium hydrogen sulfate (60 mg, 0.18 mmol) and NaHCO₃ (591 mg, 7.04 mmol) in DCM (10 mL) and water (10 mL) at 5° C. After addition, the mixture was stirred at room temperature overnight. The mixture was diluted with DCM and washed with aqueous Na₂CO₃, water, 0.5 N HCl, water. The organic layer was dried over Na₂SO₄, filtered and then the solvent was removed in vacuo to give 27-4 as a colourless oil which was used in next step without purification. A mixture of 27-4 (432 mg, 0.74 mmol), 2-hydroxy-N-phenylbenzamide (238 mg, 1.11 mmol), K₂CO₃ (153 mg, 1.11 mmol) and NaI (22 mg, 0.15 mmol) in CH₃CN (8 mL) was stirred at room temperature overnight. The mixture was diluted with water and extracted with EtOAc twice. The combined organic layers were dried over Na₂SO₄, filtered and then the solvent was removed in vacuo. The residue was purified by silica gel column chromatography (EA/PE=1/10 to 1/1) to give 27 as white solid.

### Example 28—Analysis of Prodrug Uncoupling

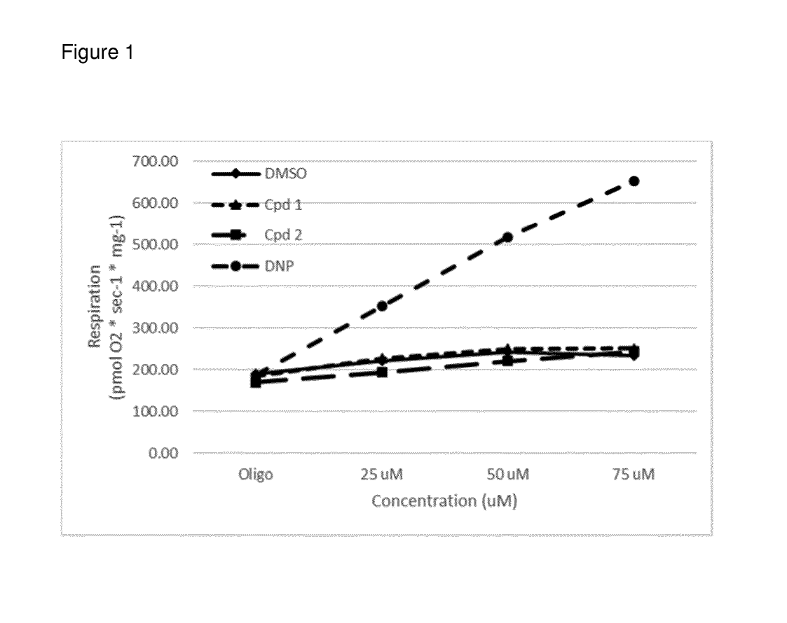

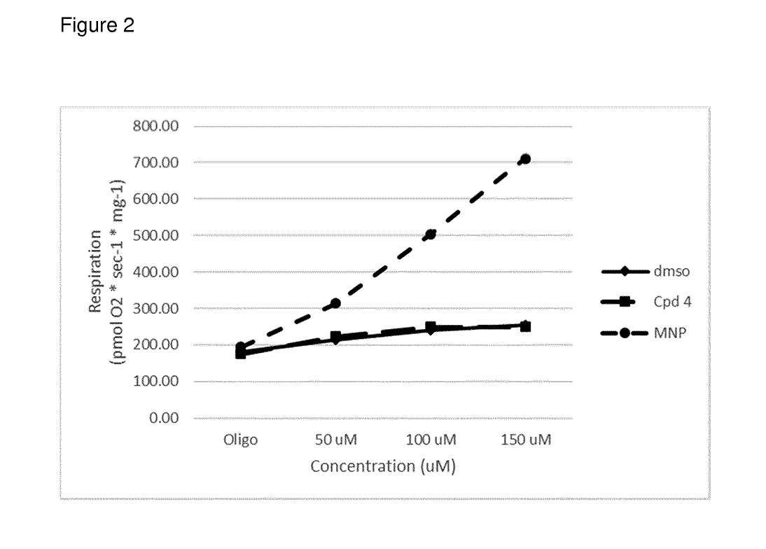

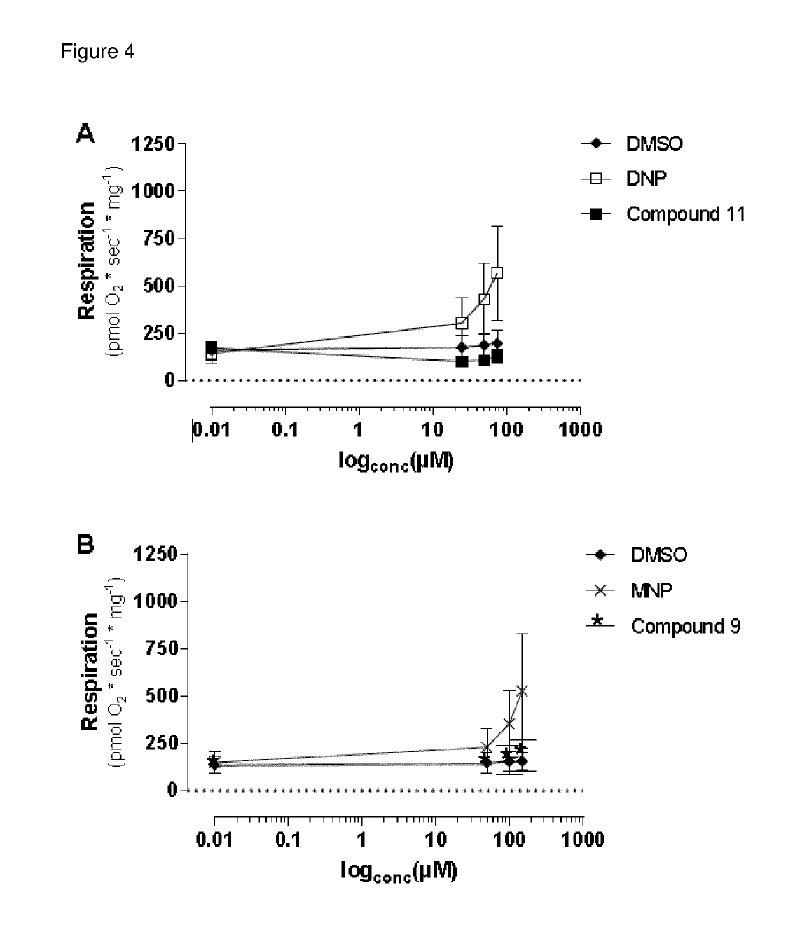

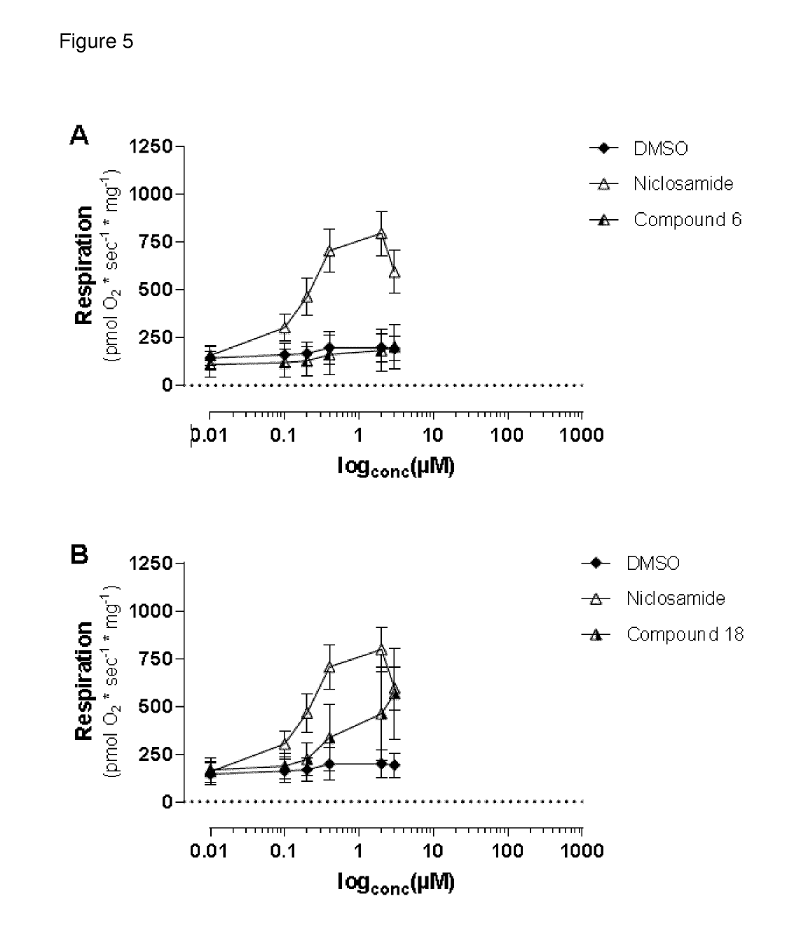

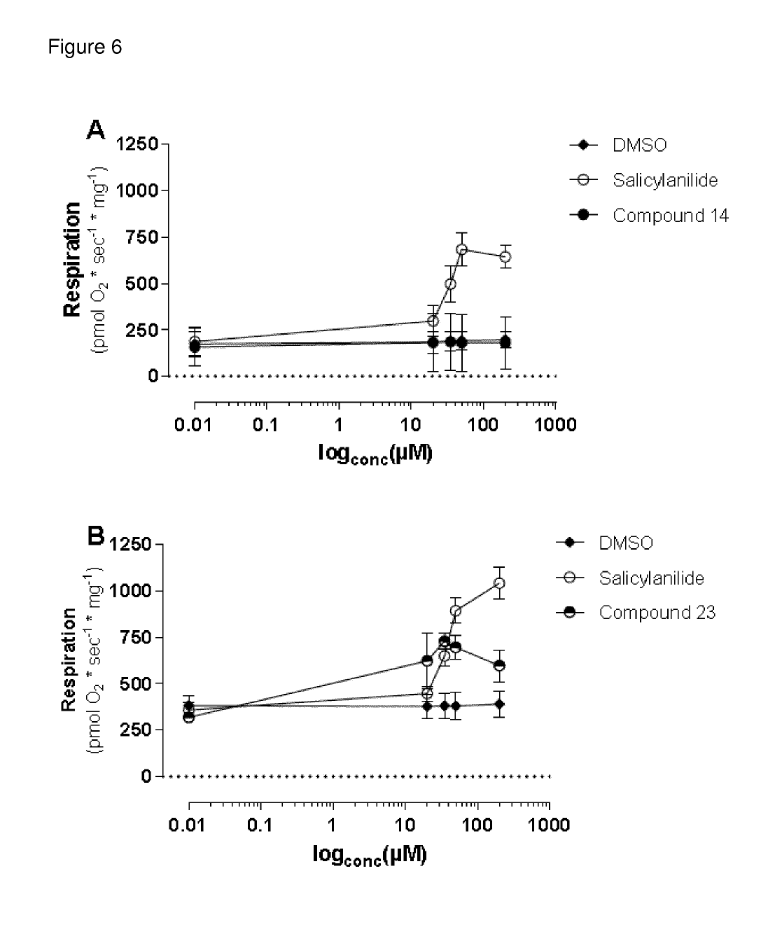

A selection of compounds of the invention were tested for free mitochondrial uncoupling and compared to known potent uncouplers DNP and MNP. The results are shown in FIGS. 1, 2, 4, 5, 6, 7, 8, 9, 10, 11 and 12.

As can be seen from the data, Compounds 1, 2, 4, 6, 9, 11, 14, 18 and 23 show reduced, little or no uncoupling in this assay, whilst DNP, MNP and niclosamide show potent uncoupling. This shows that metabolism (such as hepatic metabolism) is required for a significant uncoupling effect, allowing for improved liver targeting.

### Example 29—Analysis of Uncoupling Activity of Salicylanilide

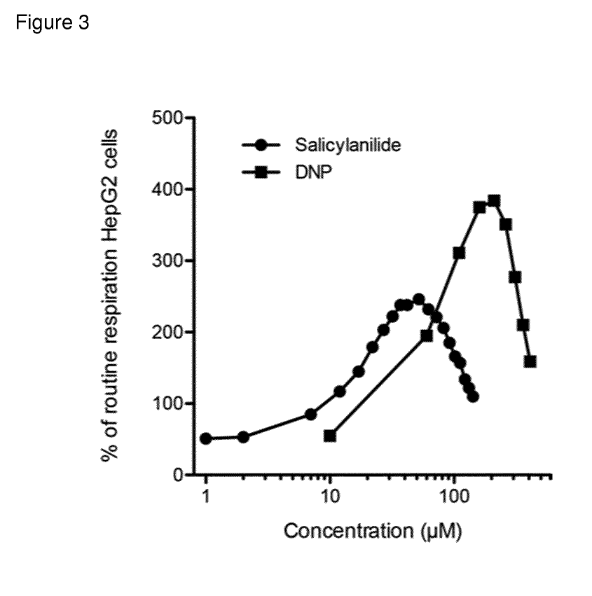

Salicylanilide and DNP were compared in a mitochondrial uncoupling assay in intact HepG2 liver cells. The results are shown in FIG. 3. As can be seen from this data, salicylanilide is more potent compared to DNP and has a lesser maximal uncoupling effect.

### Example 30—Analysis of Relative Liver Exposure Vs Extra-Hepatic Organs

As it is advantageous to have an increased ratio of liver uncoupling versus extra hepatic uncoupling, 3 mg/kg salicylanilide or 10 mg/kg compound 14 or 10 mg/kg compound 23 (which releases salicylanilide) were dosed orally to CD-1 mice and levels of salicylanilide were measured in blood, muscle and liver samples before and after dosing (see general methods). The ratio of liver vs extra-hepatic salicylanilide was then assessed, with a high ratio desirable, as this is anticipated to lead to reduced off-target uncoupling and toxicity.

| | Salicylanilide | Salicylanilide | Salicylanilide | Ratio of | Ratio of |
|---|---|---|---|---|---|
| | in liver after 1 h | in muscle after | in blood after | liver to | liver to |
| Compound | (ng/g) | 1 h (ng/g) | 1 h (ng/g) | muscle | blood |
| Salicylanilide | 694 | 7 | 14 | 99 | 50 |
| Compound 14 | 126 | 4 | BQL | 32 | N/A |
| Compound 23 | 460 | 4 | 4 | 115 | 115 |

As can be seen from the data above, salicylanilide, compounds 14 and 23 all have desirable ratios of liver to extra-hepatic exposure.

### Example 31—Comparison of Extrahepatic Uncoupling Vs Hepatic Uncoupling In Vitro

It is advantageous to have an increased level of uncoupling in hepatic tissue as compared to extra-hepatic tissue. To test for this, compounds were tested in an in vitro uncoupling assay (see Assays for evaluating mitochondrial uncoupling in intact liver cells and platelets in general methods) in HepG2 cells (hepatic) vs platelets (extra-hepatic). Data is shown in FIGS. 7, 8, 9, 10, 11 and 12. As can be seen from the data presented, compounds 6, 9, 11, 14, 18 and 23 display selective uncoupling in HepG2 cells as compared to platelets, as shown by high maximal uncoupling at low concentrations in HepG2 cells).

It may be advantageous to restrict the maximum level of uncoupling of a protonophore to a level lower than that of DNP, which is known to cause side effects and death at high doses, when tested on HepG2 cells.

### Example 32—Comparison of Permeability of Salicylanilide Vs Other Uncoupling Agents

It is advantageous to have an increased level of oral bioavailability and cellular permeability. The potential for this can be measured by a caco-2 permeability assay (see general methods). Data is show in the table below:

| | Caco-2 A-B | Caco-2 B-A | |
|---|---|---|---|
| | (Papp, | (Papp, | Caco-2 |
| Compound | 1 × 10⁻⁶ cm s⁻¹) | 1 × 10⁻⁶ cm s⁻¹) | Efflux Ratio |
| Salicylanilide | 39.5 | 33 | 0.8 |
| DNP | 24 | 27 | 1.1 |

As can be seen from the data, salicylanilide shows increased permeability and reduced efflux ratio as compared to DNP, a well-known orally bioavailable uncoupling agent.

The application of which this description and claims forms part may be used as a basis for priority in respect of any subsequent application. The claims of such subsequent application may be directed to any feature or combination of features described herein.

They may take the form of product, composition, process, or use claims and may include, by way of example and without limitation, the following claims: All references referred to in this application, including patent and patent applications, are incorporated herein by reference to the fullest extent possible.

1: A compound of Formula (I) 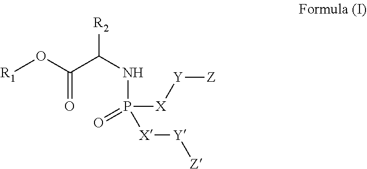
wherein:
X and X′ can independently be NH or O;
Y is absent, —CR₃R₄O—, —C(═O)O—, or 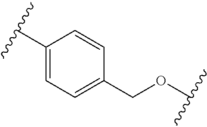 [SMILES: *OCc1ccc(*)cc1]
(X is phenyl substituent, Z connects to O);
Y′ is absent, —CR₃R₄O—, —C(═O)O—, or 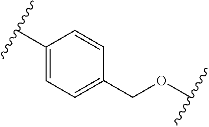 [SMILES: *OCc1ccc(*)cc1]
(X′ is phenyl substituent, Z′ connects to O);
Z is formula (II)
Z′ is CHR₂′ (C═O)OR₁′, Me, Et, iPr, Ph or formula (II)
R₁ and R₁′ are independently Me, Et, iPr, nPr, tBu, iBu, sBu or
CH₂CMe₃
R₂ and R₂′ are independently H, Me, Et, iPr, Ph, Bn
R₃ is H, Me, Et
R₄ is H, Me, Et 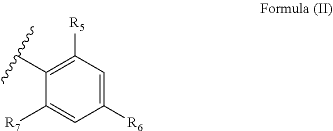
wherein:
R₅ is H, NO₂ or 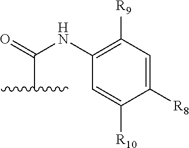
R₆ is H, NO₂, Cl, Br or I
R₇ is H, Me, Et, iPr, tBu, sBu, iBu, Cl, Br or I
R₈ is H, NO₂, Cl, Br, C(CN)H(C₆H₄)-p-Cl
R₉ is H, Cl, OH or CH₃
R₁₀ is H or Cl
R₅ and R₆ cannot both be H;
when R₆ is Cl, R₅ cannot be H or NO₂;
when Z′ is CHR₂′ (C═O)OR₁′, Me, Et, iPr then Y′ must be absent;
when Z′ is CHR₂′ (C═O)OR₁′ then X′ must be NH;
when Z′ is Me, Et or iPr then X′ must be O;
when Z is Formula II and R₆ is NO₂ then Y cannot be absent
when Z′ is Formula II and R₆ is NO₂ then Y′ cannot be absent
when Z is formula II and R₆ is NO₂ and Z′ is CHR₂′ (C═O) OR₁′
then R₂ and R₂′ cannot be H or Me;
or a pharmaceutically acceptable salt thereof.

2: The A compound according to claim 1, wherein
Z and/or Z′ are formula (II) and R₅ is

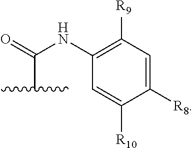

3: The A compound according to claim 1, wherein Z and/or Z′ are formula (II) and R₅ is 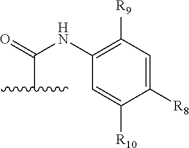
and R₆, R₇, R₈, R₉ and R₁₀ are all H.

4: The A compound according to claim 1, wherein
Z and/or Z′ are formula (II);
R₅ is 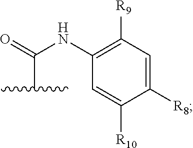
and R₆ is Cl, R₇ is H or tBu, R₈ is Cl and R₉ is NO₂, and R₁₀ is H.

5: The A compound according to claim 1, wherein Z′ is CHR₂′ (C═O) OR₁′ and Z is formula (II)
and R₅ is

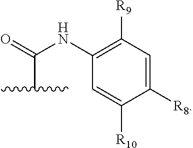

6: The A compound according to claim 1, wherein Z′ is CHR₂′ (C═O)OR₁′
R₁ and R₁′ are iPr
R₂ and R₂′ are Me or Bn
Z is formula (II)
R₅ is 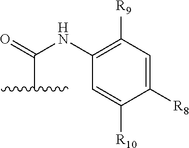
and R₆, R₇, R₈, R₉ and R₁₀ are all H.

7: The compound according to claim 1, wherein Z′ is CHR₂′ (C═O) OR₁′
R₁ and R₁′ are iPr
R₂ and R₂′ are Me or Bn
Z is formula (II)
R₅ is 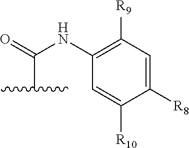
and R₆ is Cl, R₇ is H or tBu, R₈ is Cl and R₉ is NO₂, and R₁₀ is H.

8: The compound according to claim 1 having one of the following formulas:

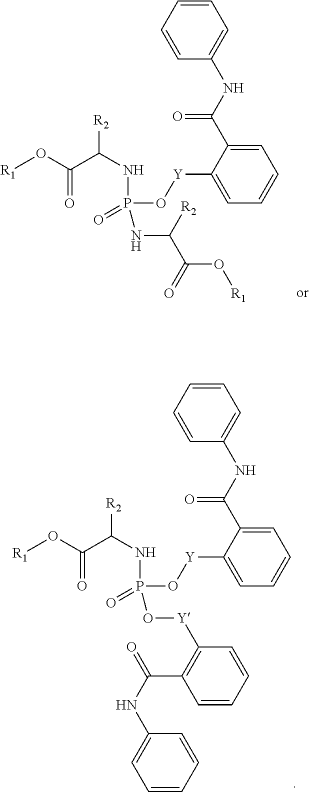

9: The compound according to claim 1, wherein the compound is selected from:

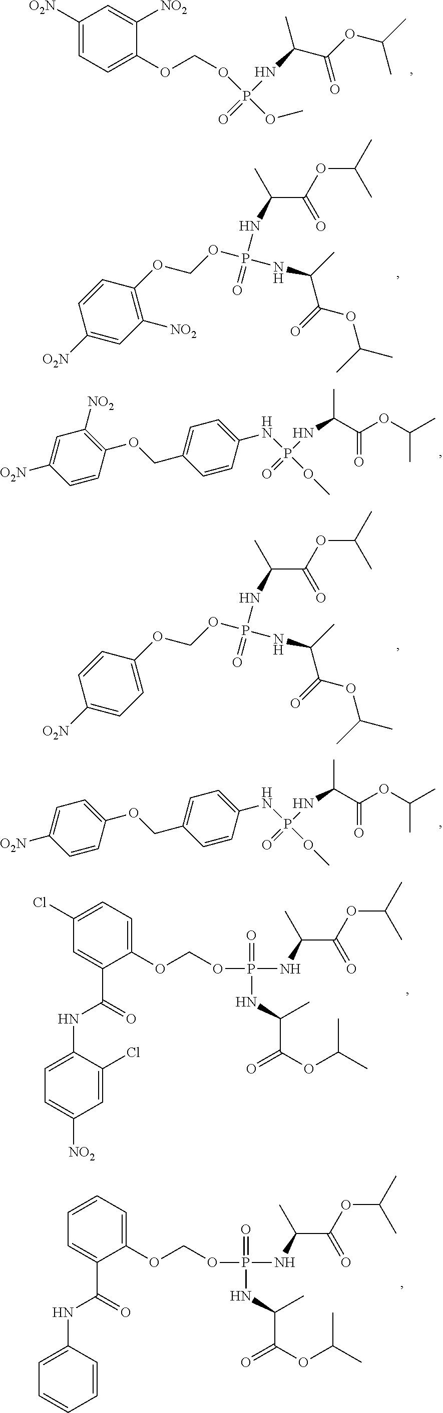

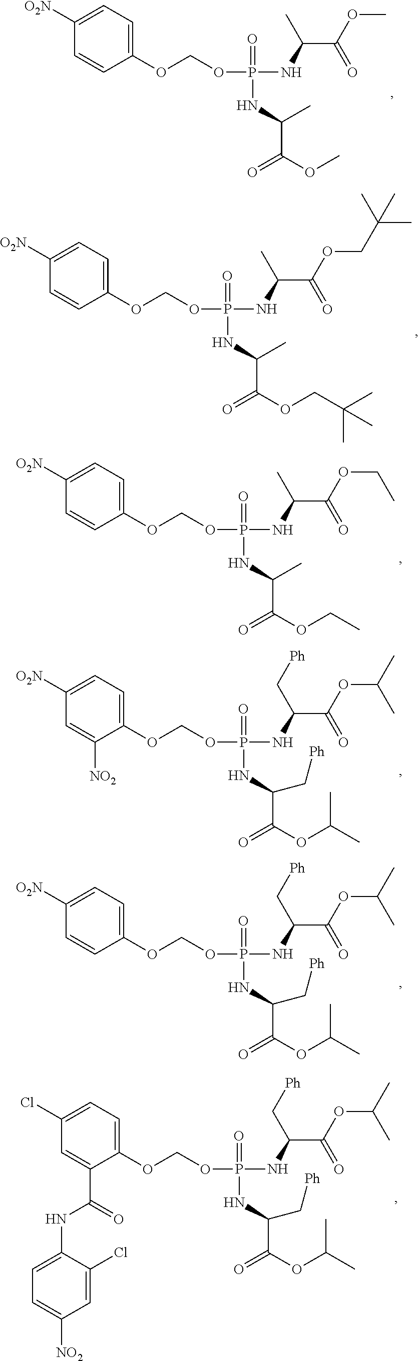

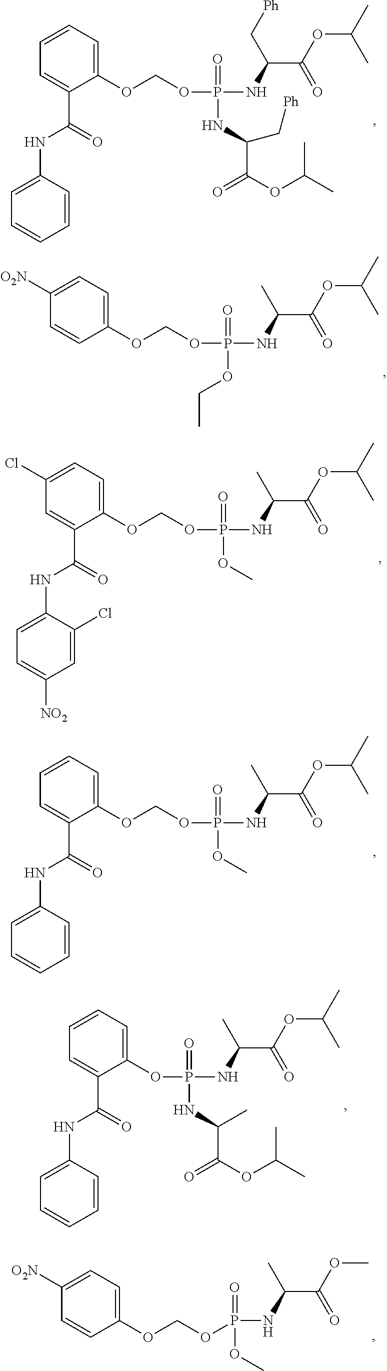

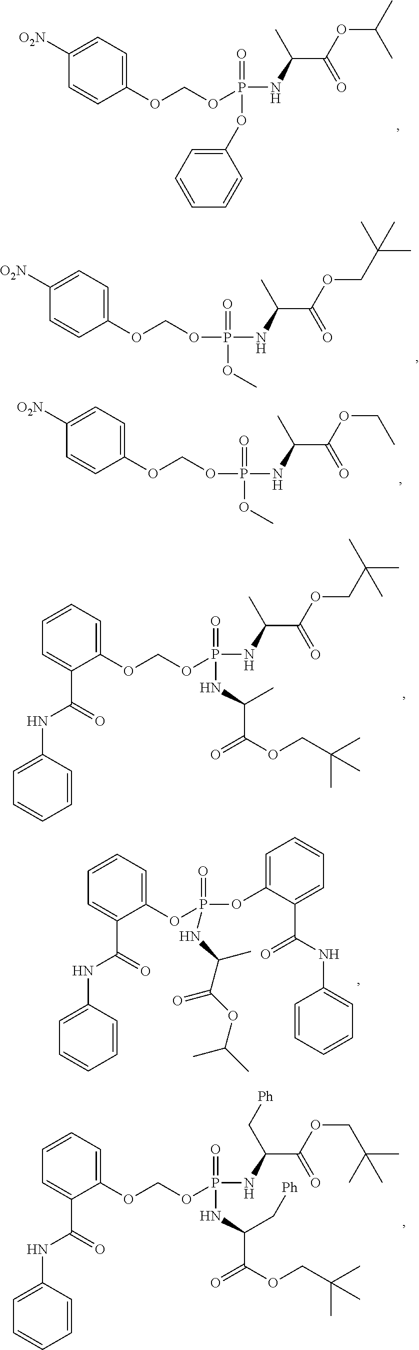

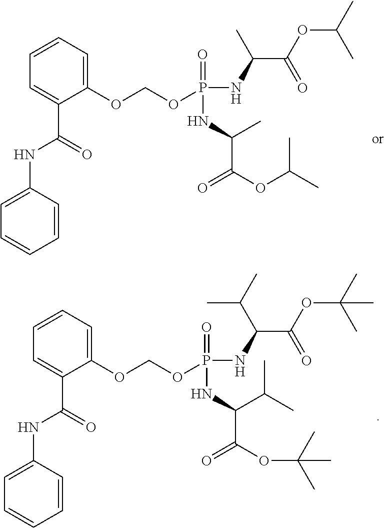 [SMILES: CC(C)OC(=O)[C@H](C)NP(=O)(N[C@@H](C)C(=O)OC(C)C)OCOc1ccccc1C(=O)Nc1ccccc1 ; CC(C)[C@H](NP(=O)(N[C@H](C(=O)OC(C)(C)C)C(C)C)OCOc1ccccc1C(=O)Nc1ccccc1)C(=O)OC(C)(C)C]

10: The compound according to claim 1 selected from

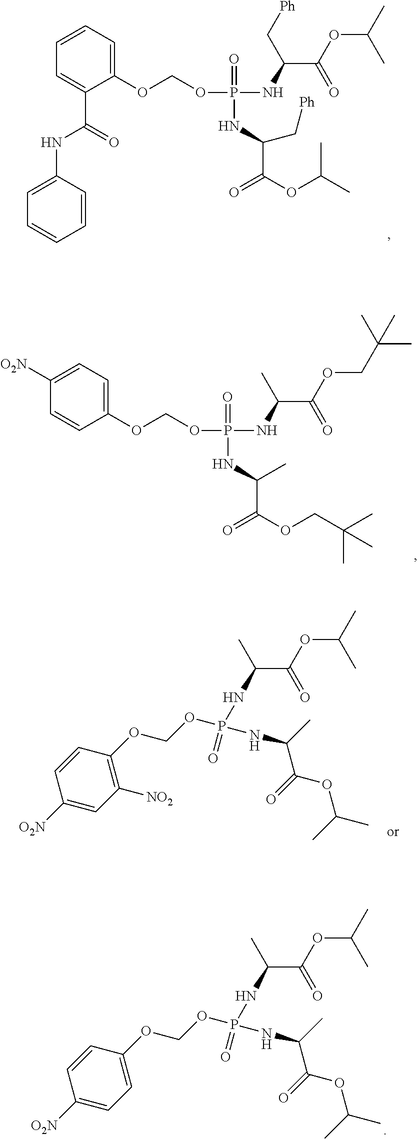 [SMILES: CC(C)OC(=O)[C@H](Cc1ccccc1)NP(=O)(N[C@@H](Cc1ccccc1)C(=O)OC(C)C)OCOc1ccccc1C(=O)Nc1ccccc1 ; C[C@H](NP(=O)(N[C@@H](C)C(=O)OCC(C)(C)C)OCOc1ccc([N+](=O)[O-])cc1)C(=O)OCC(C)(C)C ; CC(C)OC(=O)[C@H](C)NP(=O)(N[C@@H](C)C(=O)OC(C)C)OCOc1ccc([N+](=O)[O-])cc1[N+](=O)[O-] ; CC(C)OC(=O)[C@H](C)NP(=O)(N[C@@H](C)C(=O)OC(C)C)OCOc1ccc([N+](=O)[O-])cc1]

11. (canceled)

12. (canceled)

13: A method of preventing or treating a disorder or disease where liver targeted mitochondrial uncoupling is useful, the method comprising administering to the subject an effective amount of a compound according to claim 1.

14: A method of preventing or treating a disorder or disease where liver targeted mitochondrial uncoupling is useful, the method comprising administering to the subject an effective amount of salicylanilide.

15: A pharmaceutical composition comprising a compound according to claim 1 together with one pharmaceutically acceptable excipients.

16: A method of treating a subject suffering from non-alcoholic steatohepatitis (NASH) or non-alcoholic fatty liver disease (NAFLD), the method comprising administering to the subject an effective amount of a compound according to claim 1.

17: The method of claim 14, wherein said disorder or disease is non-alcoholic steatohepatitis (NASH) or non-alcoholic fatty liver disease (NAFLD).
